(19) United States
(12) Patent Application Publication
(10) Pub. No.: US 2022/0135688 A1
(43) Pub. Date: May 5, 2022

(54) FUSION PROTEIN FOR USE IN THE TREATMENT OF HVG DISEASE

(71) Applicants: Medizinische Hochschule Hannover, Hannover (DE); Technische Universität Braunschweig, Braunschweig (DE)

(72) Inventors: Elmar Jäckel, Hannover (DE); Fatih Noyan, Hannover (DE); Michael Hust, Braunschweig (DE)

(21) Appl. No.: 17/488,811

(22) Filed: Sep. 29, 2021

Related U.S. Application Data
(63) Continuation of application No. PCT/EP2020/059000, filed on Mar. 30, 2020.
Application No. PCT/EP2020/059000 is a continuation-in-part of application No. 16/310,312, filed on Dec. 14, 2018, now Pat. No. 11,160,831.

(30) Foreign Application Priority Data
Jun. 30, 2016 (EP) 16177208.2
Mar. 29, 2019 (EP) 19166421.8

(51) Int. Cl. C07K 16/28; C07K 14/47

(52) U.S. Cl. CPC C07K 16/2833; C07K 14/4702; A61K 2039/505; C07K 16/2809; C07K 16/2815; C07K 16/2818

(57) ABSTRACT

A fusion protein for use in the treatment of HvG disease in a patient having received a transplant, for use in suppressing the host's immune response directed against the transplant. The fusion protein is adapted for use in suppressing the immune rejection of a transplant which contains or expresses HLA-A*02 or SLA-01*0401 in a recipient patient who is negative for HLA-A*02 or SLA-01*0401, i.e. the patient prior to transplantation does not express HLA-A*02 or SLA-01*0401. The fusion protein is a chimeric antigen receptor (CAR), which upon expression in regulatory T-cells (T_{reg}) causes a specific suppressor activity of the regulatory T-cells in the presence of HLA-A*02 or SLA-01*0401.

### PRIORITY CLAIM

This application is a continuation of PCT Application Number PCT/EP2020/059000 filed Mar. 30, 2020, which application is incorporated by reference herein and which application claims priority from EP Application 19166421.8, filed Mar. 29, 2019; this application is also a continuation-in-part of U.S. application Ser. No. 16/310,312, which is incorporated by reference herein and which was a 371 US application of PCT/US2017/065472 filed Jun. 22, 2017 and which claims priority to EP Application number 16177208.2 filed Jun. 30, 2016.

### FIELD OF THE INVENTION

A field of the invention is fusion proteins, particularly to be used in the treatment of HvG disease in a patient having received a transplant, for use in suppressing the host's immune response directed against the transplant.

### SEQUENCE LISTING REFERENCE

The sequence listing filed via EFS web in ASCII form on Jan. 24, 2022 is incorporated by reference herein and is identical to the PDF version of the sequence listing filed via EFS web with this application on Sep. 29, 2021.

### BACKGROUND

MacDonald et al., The Journal of Clinical Investigation 1-12 (22 Mar. 2016) describes a generic CAR having an scFv domain specific for HLA-A2, and regulatory T-cells transduced for expressing the CAR for HLA-A2-specific suppression. The scFv domain contained the heavy and light chain variable regions of the monoclonal antibody BB7.2. In addition to the scFv, the CAR contained a CD28 transmembrane domain, a CD28 signalling domain and a CD3ζ signalling domain.

Inaguma et al., Gene Therapy 575-584 (2014) describe the construction of a T-cell receptor useful for directing T-lymphocytes against tumour cells that express a specific protein, comprising isolation of an antibody scFv having specificity for the tumour-specific protein when bound in a HLA-A2 complex, and using the scFv as a domain in the synthetic T-cell receptor.

Noyan et al., Cancer Gene Therapy 19, 352-357 (2012) describe induced transgene expression in hematopoietic stem and progenitor cells by lentiviral transduction for use in the treatment of solid tumours.

Galla et al., Nuc. Ac. Res. 39, 1721-1731 (2009) describe the cytotoxic effects of transposase used in transduction of cells by retroviral particles.

DiStasi et al., N Engl J Med 1673-1683 (2011) describe the genetic manipulation by introduction of sequences encoding caspase-9 dimerizer to generate a system for inducible apoptosis in the cells.

Long et al., Diabetes, 407-415 (2010) describe an assay for measuring STAT5 phosphorylation in the signalling pathway of IL-2R.

Hombach et al., Gene Therapy 1206-1213 (2010) describe a CAR for use in directing T-cells against a specific antigen, the CAR containing a modified IgG1 Fc spacer domain between an scFv and the transmembrane domain, to which transmembrane domain a signalling domain (CD28-CD3ζ) is attached.

### SUMMARY OF THE INVENTION

A chimeric antigen receptor CAR suitable for providing Treg cells with suppressor activity for an MHC class I, especially for HLA-A*02 or for CAR-SLA-01*0401, sufficiently strong to suppress the cytotoxic rejection of a transplant expressing the MHC class I, e.g. HLA-A*02 or SLA-01*0401, especially in a recipient patient who is HLA-A*02 negative, for use in the treatment of HvG disease.

### BRIEF DESCRIPTION OF THE DRAWINGS

The invention is described in greater detail by way of examples with reference to the figures, wherein

FIG. 1A schematically shows a nucleic acid construct encoding a CAR-A*02 according to the invention suitable for retroviral transduction of Treg cells,

FIG. 1B shows a schematic model of a CAR-A*02 arranged in a cell membrane,

FIG. 1C shows FACS results indicating expression of CAR-A*02 and of a comparative control CAR on the surface of cells,

FIG. 1D shows FACS results indicating that transduced Tregs express CAR-A*02 and specifically bind to HLA-A*02,

FIG. 1E show FACS results indicating that Tregs transduced with CAR-A*02 do not bind to HLA-A*01,

FIG. 1F shows FACS results for surface expression and specificity of the control CAR for PE,

FIG. 2A shows FACS results for CCR7, CD39, CD45RO, CD45RA, CTLA-4 for non-transduced Treg cells and for Treg cells expressing CAR-A*02,

FIG. 2B shows FACS results for STAT5 phosphorylation of CAR-A*02 expressing Treg cells and for non-transduced Treg cells,

FIG. 2C shows a graph representing STAT5 phosphorylation in response to IL-2 concentration,

FIGS. 3A and 3B show FACS results for activation of Treg cells expressing CAR-A*02 in response to stimulator cells,

FIGS. 3C and 3D show FACS results for proliferation and CD39 expression of Treg cells expressing CAR-A*02 or a control CAR in response to specific stimulation,

FIG. 3E shows a graph for the suppression of T-effector cells by Treg cells expressing CAR-A*02 at different cell ratios, *=P<0.05, **=P<0.01,

FIG. 4A shows results of the suppressor activity in an in vivo MLR,

FIG. 4B shows a graph of the analysis of an animal transplantation experiment using expression of the CAR-A*02 in Treg cells, and

FIG. 4C shows FACS results of an animal transplantation experiment using expression of the CAR-A*02 in Treg cells,

FIG. 5A schematically shows a nucleic acid construct encoding a CAR-SLA-01*0401 according to the invention, containing an optional fusion with P2A-FOXP3 and an additional IRES encoding ΔLNGFR as a reporter peptide, suitable for retroviral transduction of Treg cells,

FIG. 5B shows a schematic model of a CAR-SLA-01*0401 arranged in a cell membrane,

FIG. 5C shows FACS results of SC-1 cells for surface expression of CAR-SLA-01*0401,

FIG. 5D shows FACS results of SC-1 cells for expression of FOXP3 after transduction with a nucleic acid construct encoding the fusion of FIG. 5A,

FIG. 6 shows FACS results of human and porcine PBMC stained with labelled soluble scFv,

FIGS. 7A and 7B show FACS results of hybridoma cells transduced to express CAR-SLA-01*0401, analysed for NFAT signalling.

### DETAILED DESCRIPTION OF THE PREFERRED EMBODIMENTS

The present invention relates to a fusion protein for use in the treatment of HvG disease in a patient having received a transplant, for use in suppressing the host's immune response directed against the transplant. The fusion protein is adapted for use in suppressing the immune rejection of a transplant which contains or expresses an MHC class I molecule, which is the human HLA-A*02 in a recipient patient who is negative for HLA-A*02, i.e. the patient prior to transplantation does not express HLA-A*02, or the fusion protein is adapted for use in suppressing the immune reaction of a transplant which contains or expresses an MHC class I molecule, which is the porcine SLA-01*0401 (swine leucocyte antigen 01*0401). The fusion protein is a chimeric antigen receptor (CAR-A*02 or CAR-SLA-01*0401), which upon expression in regulatory T-cells (T_{reg}) causes a specific suppressor activity of the regulatory T-cells in the presence of HLA-A*02 or in the presence of SLA-01*0401, respectively. It is an advantage of the CAR-A*02 and CAR-SLA-01*0401 of the invention that the suppressor activity is limited to the transplant and results in the suppression of cytotoxic T-cells within the transplant, including cytotoxic T-cells directed against the transplant expressing HLA-A*02 or SLA-01*0401, respectively. The transplant is a solid tissue.

The fusion protein comprises or consists of a single-chain variable fragment antibody domain (scFv), a modified hCD8 hinge, a hCD8 transmembrane domain, an intracellular hCD28 signalling domain and an intracellular hCD3ζ (hCD3 zeta) signalling domain, which preferably are linked to one another from N-terminus to C-terminus, more preferably directly linked to one another form N-terminus to C-terminus.

The fusion protein can, especially in the CAR-SLA-01*0401, contain a hΔFc IgG domain in the alternative to a modified hCD8 hinge, and/or a fusion hCD28 transmembrane domain—hCD28/CD3 zeta domain in the alternative to a hCD8 transmembrane domain, an intracellular hCD28 signalling domain and an intracellular hCD3ζ (hCD3 zeta) signalling domain.

Further, the invention relates to a nucleic acid sequence encoding the CAR fusion protein, preferably contained in a viral vector, e.g. between a 5′LTR and a 3′LTR, the nucleic acid sequence more preferably being contained in a viral particle suitable for transducing Treg cells. Therein, the nucleic acid sequence and a viral vector, preferably contained in a viral particle, are for use in the treatment of Host-versus-Graft (HvG) disease. According to the invention, the treatment of HvG disease generally is the suppression of cytotoxic T-cell activity directed against the transplanted graft.

Further, the invention relates to an in vitro method for introducing suppressor activity specific for HLA-A*02 or SLA-01*0401, respectively, by expressing the fusion protein according to the invention in Treg cells, e.g. by introducing a nucleic acid sequence encoding the fusion protein CAR-A*02 or CAR-SLA-01*0401 into Treg cells, which Treg cells generally do not contain or express HLA-A*02 nor SLA-01*0401, and preferably the Treg cells are homogeneic to the patient, e.g. obtained and isolated from a biopsy of the patient. The invention provides a method of treatment of HvG disease, comprising the administration of Treg cells expressing the fusion protein CAR-A*02 or the fusion protein CAR-SLA-01*0401 according to the invention to the patient.

The invention provides a fusion protein which is chimeric antigen receptor (CAR), especially CAR-A*02 which contains an scFv domain that is specific for the human HLA-A*02 or CAR-SLA-01*0401 which contains a scFv domain that is specific for the porcine SLA-01*0401, which CAR-A*02 or CAR-SLA-01*0401 is for use in the treatment of HvG disease, e.g. for use in the treatment of cytotoxic rejection reactions in transplant recipient patients. The CAR-A*02 provides for suppression of cytotoxic T-cells when the CAR-A*02 is expressed in human Treg cells in the presence of the human HLA-A*02. The CAR-A*02, when expressed in a human Treg cell, and respectively the human Treg cell expressing the CAR-A*02, is a pharmaceutical compound for the treatment of HvG disease. Correspondingly, the CAR-SLA-01*0401 when expressed in human Treg cells, and respectively the human Treg cell expressing the CAR-SLA-01*0401 is a pharmaceutical compound for the treatment of HvG disease, when the transplanted tissue is of porcine origin and expresses SLA-01*0401. The CAR-SLA-01*0401 upon expression in a Treg cell of the transplant recipient has the advantage of suppressing the HvG directed against a transplant of porcine origin. A transplant of porcine origin can e.g. be pancreatic tissue, preferably islets.

Optionally, a Treg cell expressing the CAR-A*02 or CAR-SLA-01*0401 is genetically manipulated to also express FOXP3, preferably constitutively, e.g. by introduction of an expression cassette encoding human FOXP3 concurrent with introducing the nucleic acid sequence encoding the CAR-A*02 into a Treg cell. Further optionally, in addition to or in the alternative to genetic manipulation of a Treg cell to express FOXP3, a Treg cell in addition to expressing the CAR-A*02 or CAR-SLA-01*0401 can be genetically manipulated to express a caspase-9 dimerizer system (e.g. as described by DiStasi et al., N Engl J Med 1673-1683 (2011)) for depletion of the Treg cells following transfer into a patient.

Expression of FOXP3 with the fusion protein CAR-A*02 or CAR-SLA-01*0401, respectively, can be by expression of a fusion of P2A with C-terminally fused FOXP3 in one unified fusion protein, e.g. directly fused to the C-terminus of CAR-A*02 or CAR-SLA-01*0401, respectively. Such a fusion, e.g. encoded by one expression cassette adapted to generate one unified mRNA encoding the fusion protein of CAR-P2A-FOXP3, would yield free FOXP3 by its hydrolysis from the P2A domain. An exemplary fusion of P2A-FOXP3 is SEQ ID NO: 22, which can directly be fused to the C-Terminus of the hCD3ζ domain of the fusion protein.

The CAR-A*02 or CAR-SLA-01*0401, respectively, is a fusion protein comprising or consisting of a single-chain variable fragment antibody domain (scFv), a hinge, a transmembrane domain, an intracellular hCD28 signalling domain and an intracellular CD3 signalling domain, also termed hCD3 (hCD3 zeta) domain, which domains preferably are linked directly to one another from N-terminus to C-terminus. In the scFv domain, a variable light chain of an antibody is connected by a hinge region to a variable heavy chain of an antibody. The CAR-A*02 is characterized by its scFv domain being selected from the amino acid sequences of one of SEQ ID NO: 1 to SEQ ID NO: 12, or SEQ ID NO: 26, or SEQ ID NO: 28, the CD8 hinge and CD8 transmembrane domain preferably has an amino acid sequence of SEQ ID NO: 13, the CD28 signalling domain preferably has an amino acid sequence of SEQ ID NO: 14, and the CD3 signalling domain has an amino acid sequence of SEQ ID NO: 15. The CAR-SLA-01*0401 is characterized by its scFv domain being selected from the amino acid sequences of one of SEQ ID NO: 16 to SEQ ID NO: 19. As the signalling domains according to the invention have a human or humanized amino acid sequence, this is herein also indicated by a “h”. The hinge and can be formed by the hΔFc IgG domain, preferably of SEQ ID NO: 20. The CD8 transmembrane domain, hCD28 signalling domain and hCD3ζ signalling domain can be exchanged for a fusion of a hCD28 transmembrane domain, hCD28 signalling domain and the hCD3ζ signalling domain, preferably of SEQ ID NO: 21. Especially for the CAR-A*02 having the scFv of SEQ ID NO: 26 or the scFv of SEQ ID NO: 28, it is preferred that the signal or leader peptide has SEQ ID NO: 24

The CAR-A*02 of the invention has SEQ ID NO: 26 as its scFv domain, which may also be designated ELFA*2 (Gln Val Gln Leu Val Gln Ser Gly Gly Gly Val Val Gln Pro Gly Gly Ser Leu Arg Val Ser Cys Ala Ala Ser Gly Val Thr Leu Ser Asp Tyr Gly Met His Trp Val Arg Gln Ala Pro Gly Lys Gly Leu Glu Trp Val Ala Phe Ile Arg Asn Asp Gly Ser Asp Lys Tyr Tyr Ala Asp Ser Val Lys Gly Arg Phe Thr Ile Ser Arg Asp Asn Ser Glu Lys Thr Val Ser Leu Gln Met Ser Ser Leu Arg Ala Glu Asp Thr Ala Val Tyr Tyr Cys Ala Lys Asn Gly Glu Ser Gly Pro Leu Asp Tyr Trp Tyr Leu Asp Leu Trp Gly Arg Gly Thr Leu Val Thr Val Ser Ser Gly Gly Gly Gly Ser Gly Gly Gly Gly Ser Gly Gly Gly Gly Ser Thr Asp Val Val Met Thr Gln Ser Pro Ser Ser Leu Ser Ala Ser Val Gly Asp Arg Val Thr Ile Thr Cys Gln Ser Ser Leu Asp Ile Ser His Tyr Leu Asn Trp Tyr Gln Gln Lys Pro Gly Lys Ala Pro Lys Leu Leu Ile Tyr Asp Ala Ser Asn Leu Glu Thr Gly Val Pro Ser Arg Phe Ser Gly Ser Gly Ser Gly Thr His Phe Thr Phe Thr Ile Ser Ser Leu Gln Pro Glu Asp Phe Ala Thr Tyr Tyr Cys Gln Gln Tyr Asp Asn Leu Pro Leu Thr Phe Gly Gly Gly Thr Lys Leu Glu Ile Lys). In a variant, ELFA*2 has SEQ ID NO: 28 (Gln Val Gln Leu Val Gln Ser Gly Gly Gly Val Val Gln Pro Gly Gly Ser Leu Arg Val Ser Cys Ala Ala Ser Gly Val Thr Leu Ser Asp Tyr Gly Met His Trp Val Arg Gln Ala Pro Gly Lys Gly Leu Glu Trp Val Ala Phe Ile Arg Asn Asp Gly Ser Asp Lys Tyr Tyr Ala Asp Ser Val Lys Gly Arg Phe Thr Ile Ser Arg Asp Asn Ser Glu Lys Thr Val Ser Leu Gln Met Ser Ser Leu Arg Ala Glu Asp Thr Ala Val Tyr Tyr Cys Ala Lys Asn Gly Glu Ser Gly Pro Leu Asp Tyr Trp Tyr Leu Asp Leu Trp Gly Arg Gly Thr Leu Val Thr Val Ser Ser Gly Gly Gly Gly Ser Gly Gly Gly Gly Ser Gly Gly Gly Gly Ser Asp Val Val Met Thr Gln Ser Pro Ser Ser Leu Ser Ala Ser Val Gly Asp Arg Val Thr Ile Thr Cys Gln Ser Ser Leu Asp Ile Ser His Tyr

Leu Asn Trp Tyr Gln Gln Lys Pro Gly Lys Ala Pro Lys Leu Leu Ile Tyr Asp Ala Ser Asn Leu Glu Thr Gly Val Pro Ser Arg Phe Ser Gly Ser Gly Ser Gly Thr His Phe Thr Phe Thr Ile Ser Ser Leu Gln Pro Glu Asp Phe Ala Thr Tyr Tyr Cys Gln Gln Tyr Asp Asn Leu Pro Leu Thr Phe Gly Gly Gly Thr Lys Leu Glu Ile Lys). Optionally, at the N-terminus of ELFA*2, a signal peptide (Met Ala Leu Pro Val Thr Ala Leu Leu Leu Pro Leu Ala Leu Leu Leu His Ala Ala Ala Pro, SEQ ID NO: 24) is arranged, and encoded on a nucleic acid sequence encoding the CAR-A*02. The CAR-A*02 containing SEQ ID NO: 26 as its scFv domain, when expressed in a human Treg cell, and respectively the human Treg cell expressing this CAR-A*02, is a pharmaceutical compound for the treatment of HvG disease. The scFv of SEQ ID NO: 26, and respectively of SEQ ID NO: 28, contains, from N-terminus to C-terminus, a heavy chain variable region (VH1), a linker, and a light chain variable region (VL kappa). In the heavy chain portion and in the light chain (kappa) portion, the complementary determining regions (CDR) are marked by underlining and bold face.

In the heavy chain portion, CDR1 is Gly Val Thr Leu Ser Asp Tyr Gly (SEQ ID NO: 26, amino acids No. 26 to 33), CDR2 is Ile Arg Asn Asp Gly Ser Asp Lys (SEQ ID NO:26, amino acids No. 51 to 58), and CDR3 is Ala Lys Asn Gly Glu Ser Gly Pro Leu Asp Tyr Trp Tyr Leu Asp Leu (SEQ ID NO: 26, amino acids No. 97 to 112).

In the light chain portion, CDR1 is Leu Asp Ile Ser His Tyr (SEQ ID NO: 26, amino acids No. 166 to 171), CDR2 is Asp Ala Ser (SEQ ID NO: 26, amino acids No. 189 to 191), and CDR3 is Gln Gln Tyr Asp Asn Leu Pro Leu Thr Phe (SEQ ID NO: 26, amino acids No. 228 to 237).

Optionally, an additional Arginine is arranged at the C-terminus of SEQ ID NO: 26 or SEQ ID NO: 28.

Particularly, the scFy of a CAR of the invention
may have a heavy chain encoded by:
(SEQ ID NO. 29)
caggtgcagc tggtgcagtc tgggggaggc gtggtccagc
ctggggggtc cctgagagtc tcctgtgcag cgtctggagt
caccctcagt gattatggca tgcattgggt ccgccaggct
ccaggcaagg ggctggagtg ggtggctttt atacggaatg
atggaagtga taaatattat gcagactccg tgaagggccg
attcaccatc tccagagaca actccgagaa aacagtgtct
ctgcaaatga gcagtctcag agctgaagac acggctgtgt
attactgtgc gaaaaatggc gaatctgggc ctttggacta
ctggtacctc gatctctggg gccgtggcac cctggtcacc
gtgtcgag;

and a light chain encoded by:

(SEQ ID NO. 30)
gatgttgtga tgactcagtc tccatcctcc ctgtctgcat
ctgtaggaga cagagtcacc atcacttgcc agtcgagtct
ggacattagc cactatttaa attggtatca gcagaaacca
gggaaagccc ctaagctcct gatctacgat gcatccaatt
tggaaacagg ggtcccgtca aggttcagtg gaagtggatc
tgggacacac tttactttca ccatcagcag cctgcagcct
gaagattttg caacatatta ctgtcaacag tatgataatc
tccccctcac tttcggcgga gggaccaagc tggagatcaa
acgt

Thus, the scFv of the CAR of the invention may comprise a heavy chain and a light chain (e.g., as set out in SEQ ID NO. 26 (scFv additionally comprising a linker)), particularly where the heavy chain is encoded by SEQ ID NO. 29 (AF163303) and the light chain is encoded by SEQ ID NO. 30 (AF163304), respectively.

The CAR-A*02 fusion protein has the advantage that it is highly specific for the HLA-A*02. The CAR-SLA-01*0401 fusion protein has the advantage that it is highly specific for the SLA-01*0401. Its expression in Tregs leads to an enhancement of Treg proliferation in the presence of HLA-A*02 or SLA-01*0401, respectively, and results in increased Teff (effector T-cell) suppressor activity.

The fusion protein CAR-A*02 or CAR-SLA-01*0401 can be expressed in a Treg from a nucleic acid sequence encoding the fusion protein in an expression cassette. Optionally, the expression cassette encoding the fusion protein CAR-A*02 or CAR-SLA-01*0401 is contained in a viral vector for introduction of the nucleic acid sequence, e.g. by transduction using a viral particle containing the viral vector.

For in vitro production of Treg cells expressing the CAR-A*02, Treg cells originating from the recipient of the transplant, who can be a future recipient or a recipient having received a transplant, are used preferably. Treg cells are CD4+, CD25high and CD127low and have to be isolated from HLA-A*02 negative patients, e.g. from a blood sample by cell sorting, e.g. using FACS or magnetic beads with specific antibodies. For in vitro production of Treg cells expressing the CAR-SLA-01*0401, Treg cells originating from the recipient of the transplant, who can be a future recipient or a recipient having received a transplant, are used preferably. Treg cells are CD4+, CD25high and CD127low and have to be isolated e.g. from a blood sample by cell sorting, e.g. using FACS or magnetic beads with specific antibodies

It is an advantage of the Treg cells expressing the CAR-A*02 or CAR-SLA-01*0401 that prior to introduction into the patient and respectively following the introduction of the nucleic acid sequence encoding the CAR-A*02 or the CAR-SLA-01*0401, no in vitro expansion is necessary prior to introducing these Treg cells into a patient. For example, no in vitro expansion comprising cultivation of these Treg cells in the presence of stimulating agents in the cultivation medium is carried out in the process for producing these Treg cells. Preferably, following introduction of the nucleic acid sequence encoding the CAR-A*02 or CAR-SLA-01*0401, the Treg cells are kept in culture for about 24 h to allow expression of the CAR-A*02 or CAR-SLA-01*0401, followed by cell sorting to isolate Treg cells expressing the CAR-A*02 or CAR-SLA-01*0401. In this culture, no stimulating agents for expansion are present in the culture medium, e.g. no anti-CD3 or anti-CD28 antibodies. In this culture, the culture medium contains low dose IL-2, e.g. at 50 U/mL medium, in order to keep the Treg cells from dying. It has been found that Treg cells expressing the CAR-A*02 or the CAR-SLA-01*0401 are effective in migrating to the transplant and effective in suppressing a cytotoxic response directed against the transplant and that the Treg cells expressing the CAR-A*02 or respectively CAR-SLA-01*0401 have a stable suppression activity.

The scFv domain of the CAR-A*02 is very specific for the HLA-A*02, and to-date, no cross-reactivity or off-toxicity was found. Further, no intrinsic activity or self-activation of the CAR-A*02 was found, excluding a suppressive activity independent from the presence of HLA-A*02. The suppressive activity of Treg cells expressing the CAR-A*02 was found to be drastically higher than the suppressive activity of naïve Treg (nTreg) cells.

The CAR-A*02, or respectively CAR-SLA-01*0401, can be used in a process for producing Treg cells having suppressive activity in the presence of HLA-A*02 or respectively SLA-01*0401 by introducing a coding sequence for CAR-A*02 or respectively CAR-SLA-01*0401 into Treg cells originating from the donor prior to contact of the donor with HLA-A*02 or respectively SLA-01*0401, or into Treg cells originating from the donor following contact of the donor to HLA-A*02 or respectively SLA-01*0401, e.g. from the recipient of the transplant following transplantation.

Generally preferred, the fusion protein at its N-terminus comprises a leader peptide for secretion of the fusion protein to facilitate transmembrane transport of the scFv domains and arrangement of the transmembrane (TM) domain across the cell membrane. An exemplary leader sequence is SEQ ID NO: 24, preferred for the CAR-A*02, or SEQ ID NO: 25, preferred for the CAR-SLA-01*0401.

The invention relates to a fusion protein comprising a single-chain variable fragment antibody domain (scFv), a hinge, a transmembrane domain, an intracellular hCD28 signalling domain and an intracellular hCD3ζ (hCD3 zeta) signalling domain forming a chimeric antigen receptor having specificity for HLA-A*02 (CAR-A*02) or having specificity for SLA-01*0401 (CAR-SLA-01*0401) for use in the treatment of HvG disease in a patient, characterized by the single-chain variable fragment antibody domain (scFv) preferably having an amino acid sequence which is selected from the group consisting of SEQ ID NO: 1 to SEQ ID NO: 12, SEQ ID NO: 26, or SEQ ID NO: 28, or SEQ ID NO: 16 to SEQ ID NO: 19. This fusion protein preferably is characterized in that the hinge and the transmembrane domain have an amino acid sequence of SEQ ID NO: 13, the hCD28 signalling domain has an amino acid sequence of SEQ ID NO: 14, and the hCD3 (hCD3 zeta) signalling domain has an amino acid sequence of SEQ ID NO: 15. This fusion protein can further be characterized in that the hinge is a hΔFc IgG domain having an amino acid sequence of SEQ ID NO: 20. This fusion protein can further be characterized in that the hinge and the transmembrane domain, which is a CD8 transmembrane domain, have an amino acid sequence of SEQ ID NO: 13, the hCD28 signalling domain has an amino acid sequence of SEQ ID NO: 14, and the hCD3 ζ domain has an amino acid sequence of SEQ ID NO: 15, or the hCD28 signalling domain including the hCD3ζ signalling domain have an amino acid sequence of SEQ ID NO: 21. This fusion protein can further be characterized in that it is expressed in a CD4+CD25+CD127low HLA-A*02 negative human regulatory T (Treg) cell. Use of this fusion protein can further be characterized in that the patient is HLA-A*02 negative and in that the patient contains or is intended to contain a solid tissue transplant which is HLA-A*02 positive. Use of the fusion protein can further be characterized in that the patient is human and contains or is intended to contain a solid tissue transplant which is SLA-01*0401 positive. This fusion protein can further be characterized in that the tissue transplant comprises porcine islet cells. This fusion protein can further be characterized in that it comprises or consists of, from N-terminal to C-terminal, one scFv domain having an amino acid sequence selected from the group consisting of SEQ ID NO: 1 to SEQ ID NO: 12, SEQ ID NO:26 or SEQ ID NO: 28, a hinge and a transmembrane domain having an amino acid sequence of SEQ ID NO: 13, a hCD28 signalling domain having an amino acid sequence of SEQ ID NO: 14, and a hCD3ζ (hCD3 zeta) signalling domain having an amino acid sequence of SEQ ID NO: 15. This fusion protein can further be characterized in that it comprises or consists of, from N-terminal to C-terminal, one scFv domain having an amino acid sequence selected from the group consisting of SEQ ID NO: 16 to SEQ ID NO: 19, a hΔFc IgG domain as a hinge having an amino acid sequence of SEQ ID NO: 20, a hCD28 transmembrane domain and a hCD28/hCD3 signalling domain having an amino acid sequence of SEQ ID NO: 21. This fusion protein can further be characterized in that it is expressed from a nucleic acid sequence encoding the fusion protein with optionally an additional N-terminal secretory leader peptide. This fusion protein can further be characterized in that it is expressed from a nucleic acid sequence encoding the fusion protein with an additional N-terminal secretory leader peptide and an additional C-terminal P2A-hFOXP3 having an amino acid sequence of SEQ ID NO: 22. This fusion protein can further be characterized in that the leader peptide has an amino acid sequence selected from SEQ ID NO: 24 and SEQ ID NO: 25. This fusion protein can further be characterized by providing suppressor activity to a CD4+CD25+CD127low HLA-A*02 negative human regulatory T (Treg) cell in the presence of HLA-A*02 positive solid tissue or in the presence of SLA-01*0401 positive solid tissue. This fusion protein can further be characterized by providing for homing capability to secondary lymphoid organs in a CD4+CD25+CD127low HLA-A*02 negative human regulatory T (Treg) cell.

Generally, the FACS results that are shown are representative of three independent experiments.

### Example 1: Retroviral Vector Encoding CAR-A*02 and Cells Expressing CAR-A*02

The scFv domains for the CAR-A*02 were generated by affinity selection for HLA-A*02 using a phage display library expressing an anti-HLA-A*02 antibody. The anti-HLA-A*02 (nucleotide sequences accessible at EBI for heavy chain: AF163303; light chain: AF163304) was cloned from a patient who had developed A*02 reactive antibodies subsequent to blood transfusion.

As a result, antibodies could be isolated which had a significantly increased affinity for HLA-A*02 compared to the originally cloned anti-HLA-A*02 antibody.

The coding sequences for the scFv domains were cloned to generate a coding sequence for one fusion protein, from N-terminus to C-terminus containing the variable light chain, a linker, the variable heavy chain, the hCD8 hinge domain, the hCD6 transmembrane domain, the hCD28 intracellular signalling domain and the hCD3ζ intracellular signalling domain. The coding sequence was cloned between a 5″-LTR and a 3″-LTR of a retroviral vector, which between this coding sequence for the CAR-A*02 and the 3″-LTR additionally contained the coding sequence for the non-signalling surface molecule ΔLNGFR (truncated low-affinity nerve growth factor) as a reporter protein under the control of an IRES (internal ribosomal entry site) element acting as a promoter. In addition to its function as a reporter for expression of the CAR-A*02, such a reporter can be used for isolation of transduced cells, e.g. by affinity isolation using antibody specific for the reporter and coupled to a carrier, e.g. to magnetic beads.

For transduction, the nucleic acid sequence encoding CAR-A*02 was cloned into a gamma-retroviral LTR-driven expression vector.

The reporter ΔLNGFR was detected by flow cytometry using anti-CD271 antibody (C40-1457, Becton Dickinson).

For introduction of nucleic acid sequences encoding the CAR-A*02, a reporter, e.g. ΔLNGFR, were contained in a retroviral vector, producing viral particles containing the vector as described by Galla et al., Nuc. Ac. Res. 39, 1721-1731 (2009). After isolation of Treg cells, which are considered nTreg cells, these were stimulated with plate-bound anti-CD3 antibody (OKT-3, 5 μg/mL) and soluble anti-CD28 antibody (CD28.2, obtained from BioLegend, 5 μg/mL) in complete medium for 48 h. Prior to transduction, protamine sulfate (4 μg/mL, obtained from Sigma) was added to the Treg cultures. Treg cells were spin-infected with retroviral particles encoding CAR-A*02 or the control CAR specific for PE at 31° C. at 700×g for 1.5 h.

After transduction with the expression vector for the control CAR, SC-1 cells could be immunologically stained for ΔLNGFR by anti-CD271 antibody and stained for scFv using anti-IgG-Fab (obtained from Jackson Lab.), demonstrating surface expression of the control CAR and recognition of PE by the control CAR. Although the SC-1 cells do not express either FOXP3 or B220, they stain positive with a PE-conjugated antibody.

Human Treg cells were isolated from human PBMC using FACS with the following antibody combinations: anti-CD8 (HIT8a, obtained from BioLegend), anti-CD4 (RPA-T4, obtained from Becton Dickinson), anti-CD25 (M-A251, obtained from Becton Dickinson), anti-CD127 (hIL-7R-M21, obtained from Becton Dickinson), resulting in isolation of CD8⁻ CD4⁺ C25^{high}, CD8⁻ CD4⁺ C25^{high} CD127^{low} Tregs with a purity of at least 90%. The PBMC preparation was produced by density gradient centrifugation over Ficoll-Paque Plus (obtained from GE Healthcare) from different HLA-typed healthy donors after ethical approval and individual written informed consent.

Treg cells were transduced as described by Galla et al., Nuc. Ac. Res. 39, 1721-1731 (2009). Generally, all T-cell cultures and all T-cell related assays were performed in complete medium (RPMI 1640 GlutaMax-I (obtained from Gibco), supplemented with 10% fetal bovine serum (FBS) (obtained from Gibco), 1% penicillin and streptomycin (obtained from Biochrom), 0.05 mM (3-mercaptoethanol (obtained from Gibco), 20 mM HEPES (obtained from Gibco), 1% sodium pyruvate (obtained from Gibco) and 500 IU/mL IL-2 (Proleukin, obtained from Novartis) in humidified incubators at 37° C. and 5% CO₂. All cell lines were tested negative for mycoplasma.

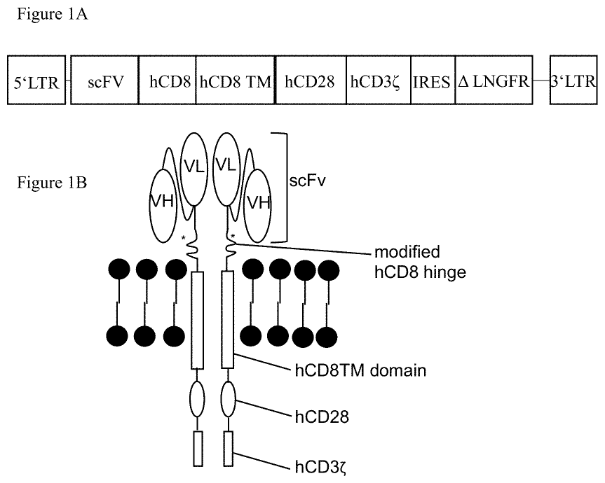

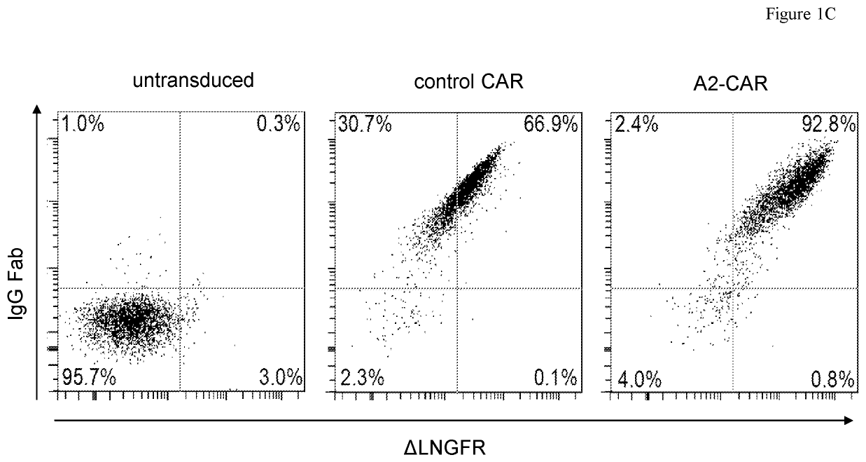

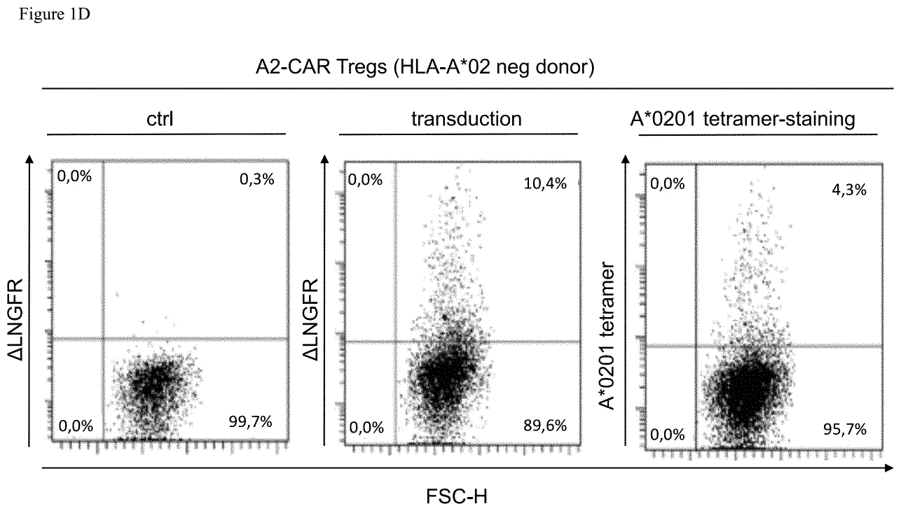

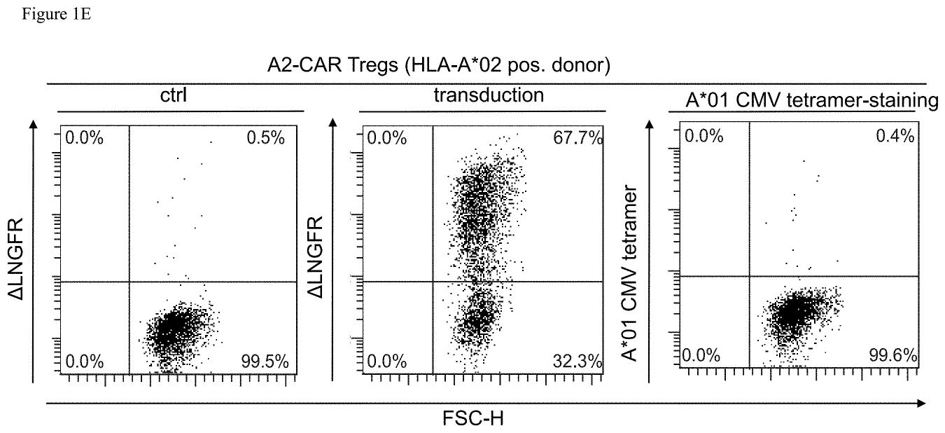

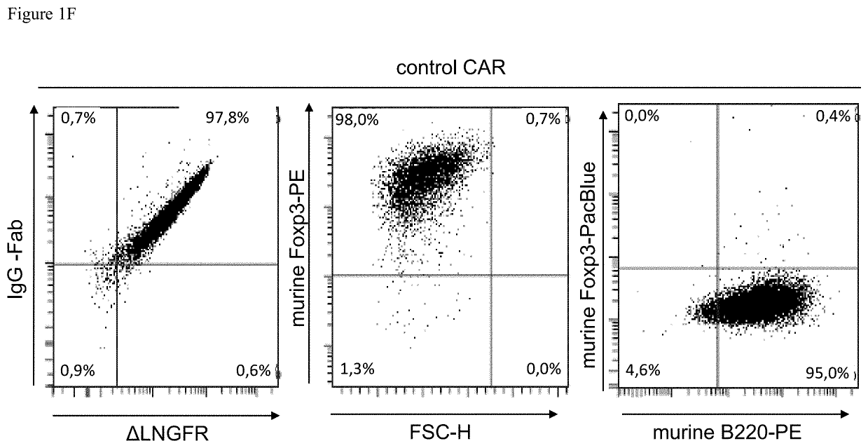

FIG. 1A shows the arrangement of the nucleic acid construct of the CAR-A*02 including the coding sequence for the reporter ΔLNGFR under the control of an IRES element flanked by a 5′-LTR and a 3″-LTR of the gamma-retroviral vector. Generally, expression of a membrane bound protein in a Treg cell, e.g. under the control of an IRES linked to the coding sequence for the CAR-A*02, preferably in a viral vector, can be used for isolation of genetically manipulated Treg cells by affinity isolation directed to the membrane bound protein. An example for such a membrane bound protein is the ΔLNGFR.

FIG. 1B shows a model of the CAR-A*02 with its transmembrane domain spanning a cell membrane and the scFv arranged on the outer cell surface and the intracellular signalling domains arranged within the cytoplasm.

Membrane-anchored expression of the fusion protein on the surface of cells was tested using transduction of hybridoma cells. In short, hybridoma cells were transduced with the retroviral vectors encoding a CAR-A*02 or the negative control fusion protein. Stimulation of the transduced hybridoma cells was by contact with various HLA-A*02 positive or HLA-A*02 negative human PBMC (peripheral blood mononuclear cells). Co-culture was for 20 h with transduced hybridoma cells and irradiated (30 Gy). For specific staining of ΔLNGFR, an anti-CD271 antibody, C40-1457 (obtained from Becton Dickinson) was used, for specific staining of the CAR-A*02, the monoclonal antibody (mAb) anti-IgG-F(ab) (obtained from Jackson Labs) was used. Analysis was generally made by flow cytometry using a flow cytometer FACSCalibur (Becton Dickinson) or a LSRII (Becton Dickinson) using the FACSDiva software and FlowJo Software (Tree Star Inc.). For statistical analysis, the GraphPad Prism version 5.0 was used.

FIG. 1C shows expression and localisation of the reporter ΔLNGFR on the surface of the transduced hybridoma cells in original hybridoma cells (untransduced), staining for ΔLNGFR (control CAR) only, and staining for CAR-A*02 (A2-CAR). The results show that both the reporter ΔLNGFR and the CAR-A*02 were expressed on the surface of the transduced hybridoma cells.

Treg cells (CD4⁺ CD25^{high} CD127^{low}) isolated from a HLA-A2*-negative person (HLA-A*02 neg donor) were transduced by the retroviral vector to express the CAR-A*02 and the reporter ΔLNGFR. For staining, HLA I tetramers displaying a hepatitis C virus peptide (HLA-A1-CMV Pentamer, obtained as pp65 from Prolmmune) were used.

The FACS results of FIG. 1D show that transduced cells were stained with the HLA A*0201 (A*0201, obtained from Beckman coulter Immunomics, San Diego, USA) tetramers.

The FACS results of FIG. 1E show that the Treg cells from a HLA-A2*-positive person (HLA-A*02 pos donor), transduced to express the CAR-A*02 and the reporter ΔLNGFR, did not stain with A*01 tetramers.

The results of FIG. 1 show that the CAR-A*02 is expressed on the surface of transduced Treg cells, and that it specifically recognizes the HLA-A*02 tetramers, e.g. not recognizing HLA-A*01 tetramers. Further, it was found that the specific staining by the A*02 tetramers was independent of the peptide bound to the A*02 tetramer.

As a negative control CAR, a fusion protein containing an scFv specific for phycoerythrin (PE) in the place of the scFv specific for HLA-A*02, but otherwise identical, was encoded in the same expression vector. For determining surface expression and specificity of the control CAR, SC-1 cells, foetal mouse embryo cells which lack host-range restrictions for murine leukemia viruses (ATCC CRL-1404) were transduced according to Noyan et al., Cancer gene therapy 19, 352-357 (2012). The specificity of the control CAR for phycoerythrin (PE) was assessed by the use of several PE-conjugated proteins and PE-conjugated antibodies: murine B220-PE (RA3-6B2, obtained from Caltag), murine Foxp3-PE and murine Foxp3-PacBlue (FJK-165, obtained from eBioscience), using the eBioscience Fix/Perm Kit for intracellular Foxp3 staining according to the manufacturer's instructions.

The FACS results of FIG. 1F show that the negative control CAR is expressed on the surface of cells and recognizes PE.

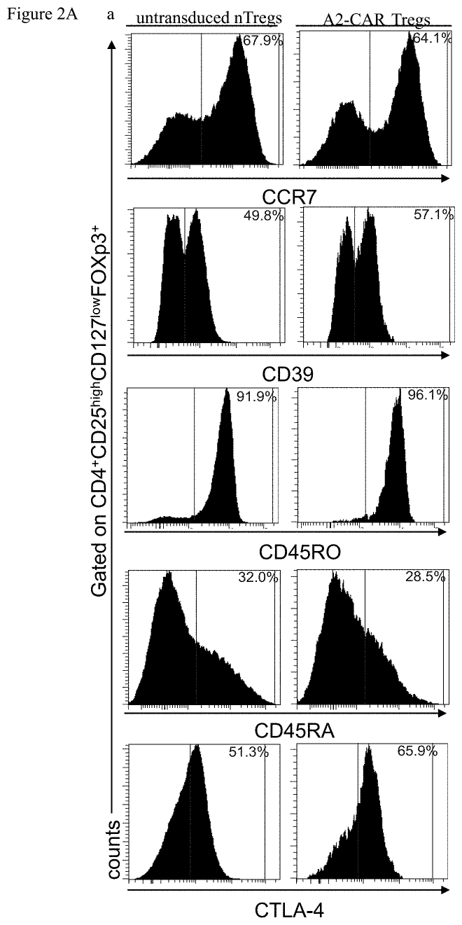

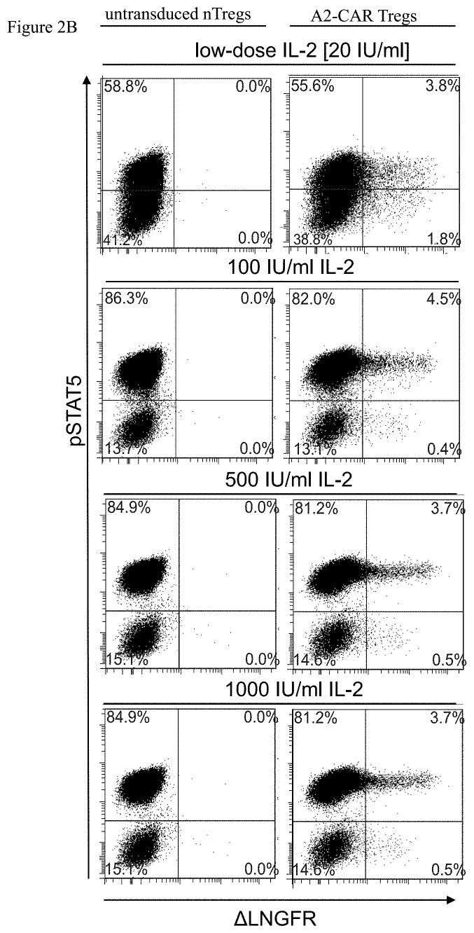

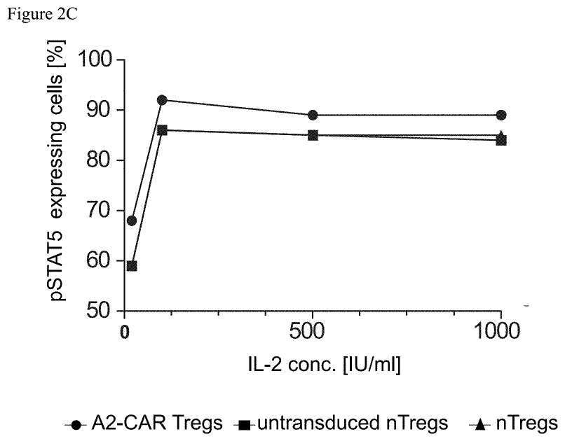

The phenotype of Treg cells that express the CAR-A*02 was analysed using Treg cells obtained from HLA-A*02 negative donors in order to prevent activation of the CAR-A*02 by the Treg cells themselves after transduction. This situation approximates the situation in a HLA-A*02 negative recipient. It was found that the transduction did essentially not affect the nTreg phenotype, with similar levels of effector molecules CTLA-4 and CD39 displayed in CAR-A*02-transduced Treg cells and in non-transduced nTreg cells, and similar percentages of CD45RA+naïve Treg cells and similar expression of CCR7 were needed for homing the cells to secondary lymphoid organs. For staining, the antibodies anti-CD39 (A1, obtained from BioLegend), anti-CD45RA (HI100, obtained from Becton Dickinson), anti-CD45RO (UCHL1, obtained from BioLegend), anti-CCR7 (3D12, obtained from Becton Dickinson), anti-CTLA-4 (BNI3, obtained from Becton Dickinson), anti-FoxP3 (PCH101, obtained from eBioscience) were used. FIG. 2A shows these FACS results. The same phenotype was found for Treg cells transduced with the PE-specific control CAR.

STAT5 phosphorylation was measured using FACS (method as described in Long et al., Diabetes, 407-415 (2010) using anti-pSTAT5 antibody (pY694, 47/SATS) obtained from Becton Dickinson) at different doses of IL-2 for Treg cells transduced with CAR-A*02 and non-transduced nTregs from the same experiment. The FACS results are depicted in FIG. 2B, showing that both these Treg cells showed high levels of STAT5 phosphorylation already under the low doses of IL-2 necessary for survival of nTreg in culture. The Treg cells transduced with CAR-A*02 showed a higher baseline and a slightly higher maximum STAT5 phosphorylation level in comparison to non-transduced cells. The graph of FIG. 2C compares STAT5 phosphorylation levels in relation to IL-2 doses. No defects in IL-2 signalling were observed. These results show that transduction of Treg cells to express CAR-A*02 did not significantly affect STAT5 phosphorylation, indicating no impairment of homing ability of these transduced cells.

For analysis of the function of CAR-A*02-transduced cells, T-cell hybridomas stably expressing a reporter construct containing an NFAT-sensitive IL-2 promoter to control GFP expression were transduced with the CAR-A*02. For detection of CAR expression in FACS analysis, the reporter ΔLNGFR was detected, NFAT stimulation was detected as expression of GFP (green fluorescent protein).

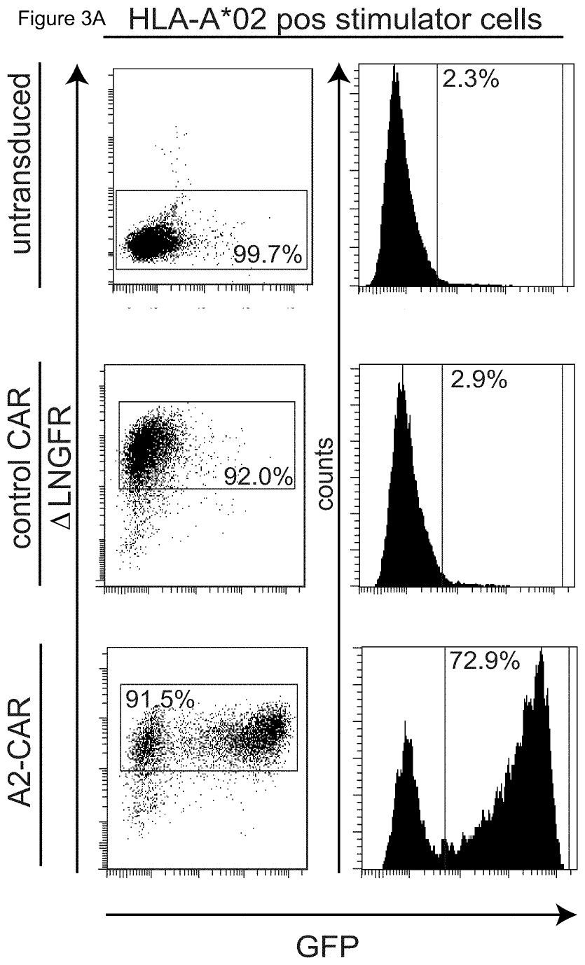

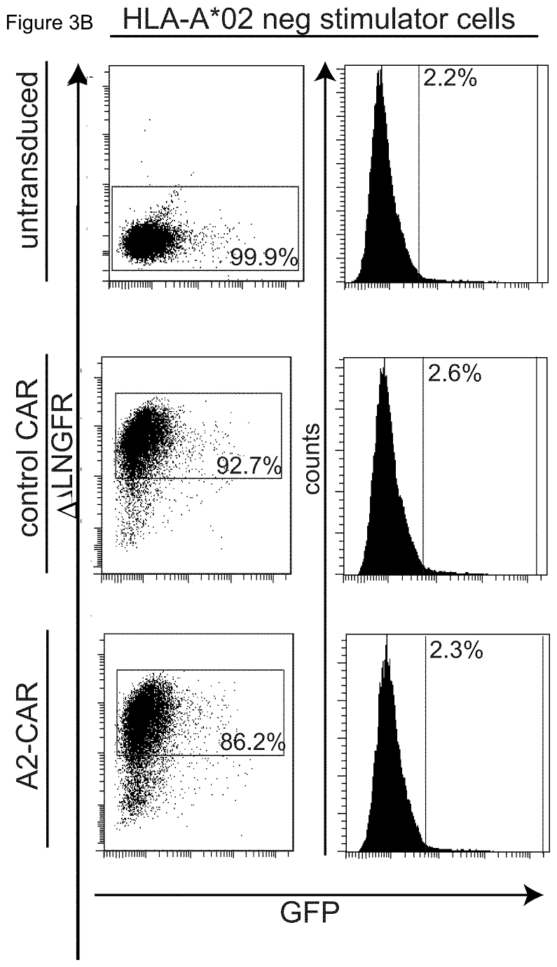

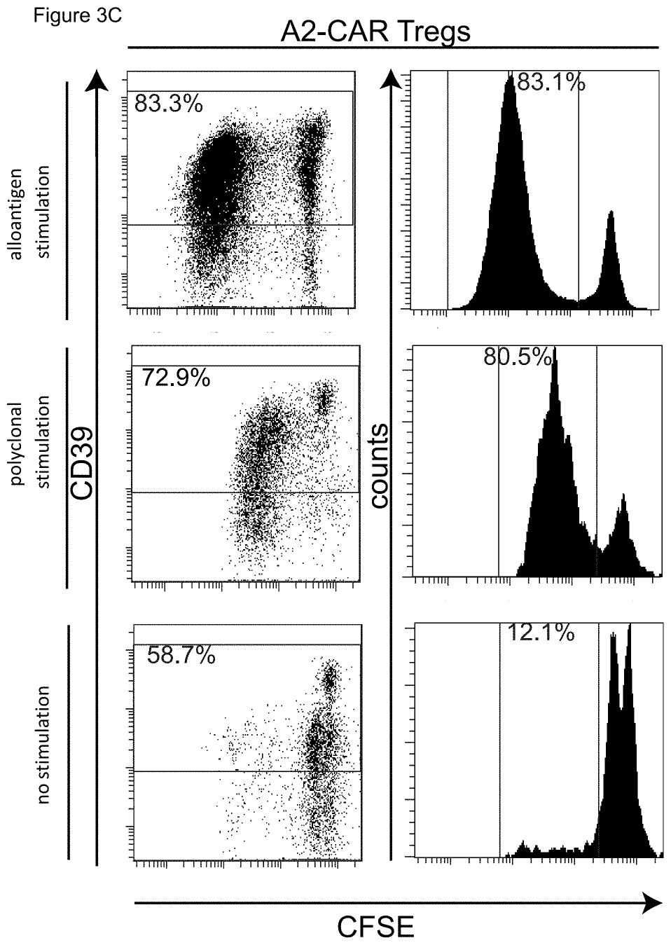

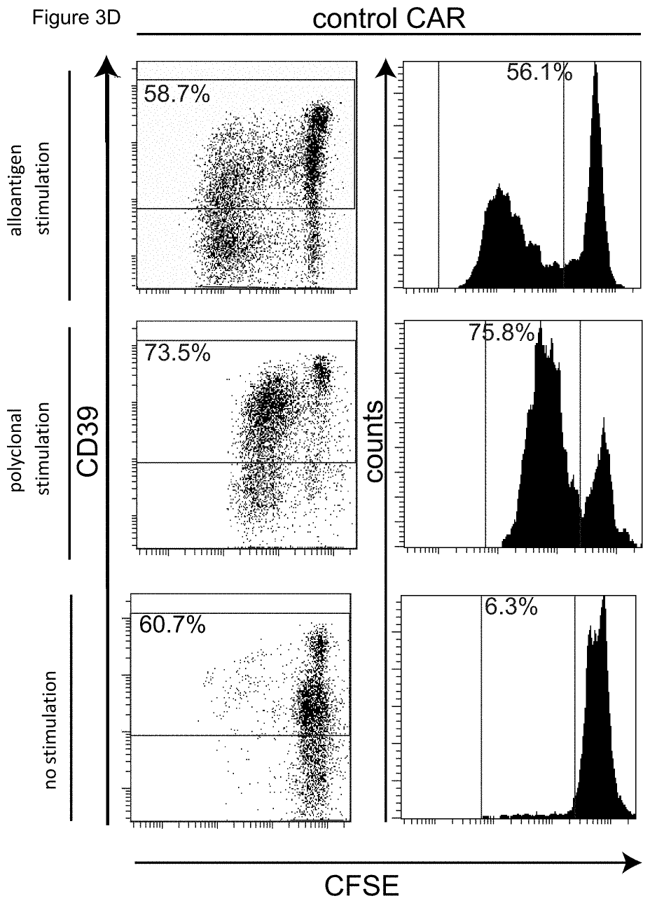

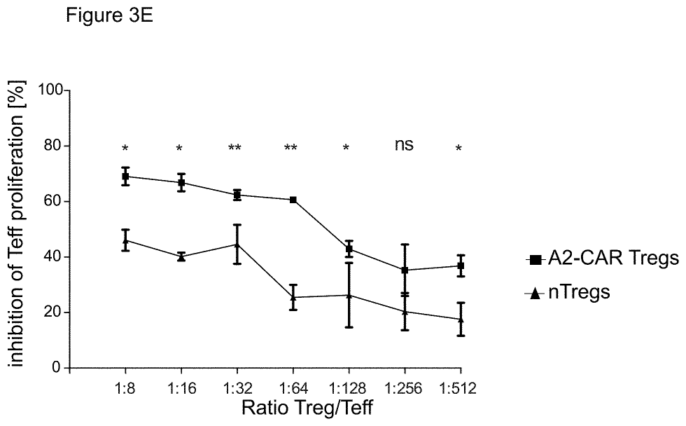

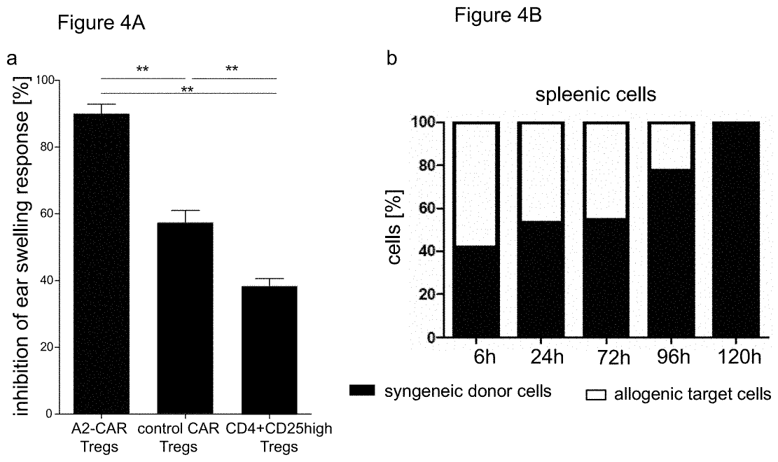

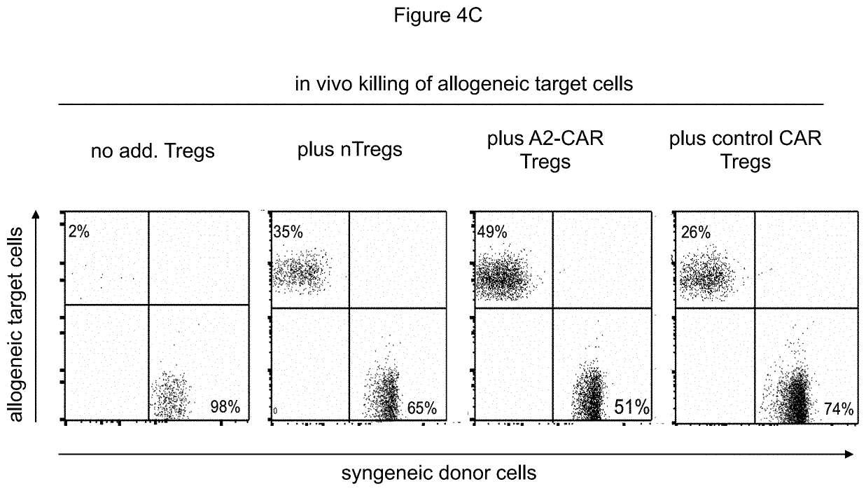

As shown in FIGS. 3A and 3B, it was found that CAR-A*02-transduced cells (A2-CAR) did not show any NFAT activation nor the associated expression of GFP after transduction, but NFAT activation and GFP expression were strongly up-regulated by co-culture with HLA-A*02+ PBMC acting as stimulator cells but not in response to HLA-A*02-PBMC. The non-transduced (untransduced) T-cell hybridomas and the cells that were transduced with the ΔLNGFR (control CAR) did not show a reaction to HLA-A*02 positive nor to HLA-A*02 negative PBMC stimulator cells. This result shows that the CAR-A*02 according to the invention is capable of signal transduction necessary to activate NFAT. As the hybridomas do not express any endogenous T-cell receptor (TCR), the signal transduction that was observed is entirely caused by the signalling of the CAR-A*02.

The differentiation between signalling by CAR or by TCR will be more difficult when HLA-A*02 negative donor Treg cells are transduced with the CAR-A*02, because 8 to 12% of these nTreg cells will have a TCR that also recognizes HLA-A*02. Therefore, the CAR-A*02 was tested against a wide panel of human PBMC presenting various MHC I and MHC II alleles, using expression in the T-cell hybridomas containing the reporter construct. The analysis was by flow cytometry of GFP expression. The results showed that the CAR-A*02 (HLA-A2 CAR) upon expression in the hybridomas recognized all HLA-A*02 positive donor samples without any cross-reactivity with HLA-A*02 negative blood samples. For comparison, the control CAR specific for PE (irrelevant CAR) was used. The results are summarized in the following table, wherein the individual HLA-A and HLA-B are indicated in each row for the numbered samples (Human PBMCs) and X designates GFP expression:

| Human | | | | | HLA-A2 | irrelevent |
|---|---|---|---|---|---|---|
| PBMCs | HLA-A | | HLA-B | | CAR | CAR |
| 1 | 2 | 23 | 44 | | X | — |
| 2 | 3 | 24 | 7 | 13 | — | — |
| 3 | 1 | 24 | 8 | 40 | — | — |
| 4 | 1 | | [symbol] | 57 | — | — |
| 5 | 3 | 24 | 7 | 13 | — | — |
| 6 | 2 | 24 | 35 | 37 | X | — |
| 7 | 24 | 31 | 13 | 51 | — | — |
| 8 | 2 | | 51 | 62 | X | — |
| 9 | 2 | | 60 | 61 | X | — |
| 10 | 2 | 3 | 38 | 44 | X | — |
| 11 | 3 | 25 | 7 | 18 | — | — |
| 12 | 2 | 25 | 35 | 44 | X | — |
| 13 | 11 | 23 | 27 | 44 | — | — |
| 14 | 2 | 31 | 62 | 27 | X | — |
| 15 | 3 | | 7 | 62 | — | — |
| 16 | 3 | 30 | 7 | 13 | — | — |
| 17 | 2 | 24 | 7 | 62 | X | — |
| 18 | 3 | | 35 | | — | — |
| 19 | 2 | 3 | 13 | 18 | X | — |
| 20 | 1 | 2 | 27 | 60 | X | — |

[symbol]

indicates data missing or illegible when filed

The hybridoma cells expressing the control CAR after co-culture with the blood samples did not express GFP (−) for any of the HLA. This demonstrated the high specificity of the CAR-A*02 according to the invention for HLA-A*02, showing low or absent unspecific or off-target activity.

The effect of activating the CAR-A*02 according to the invention when it is expressed in human HLA-A*02 negative Treg cells was tested using HLA-A*02 positive PBMC as stimulator cells. Proliferation of Treg cells expressing the CAR-A*02 was analysed based on a CFSE dilution assay, for which the Treg cells were labelled with CFSE (5 mM, obtained from Invitrogen). For the HLA-A*02 negative Treg cells expressing the CAR-A*02 polyclonal stimulation was used by co-cultivation with irradiated (30 Gy) HLA-A*02 positive PBMC (stimulator cells) which were also contacted with 5 mM APC cell proliferation dye (eFluor 670, obtained from eBioscience) in a 1:4 ratio. For the human HLA-A*02 negative Treg cells transduced to express the control CAR (specific for PE), stimulation was by anti-CD3/anti-CD28 directed to the TCR. Detection of CD39 using anti-CD39 antibody (A1, BioLegend) was measured for Treg activation, and CFSE was detected for proliferation. For comparison, FACS analysis of CFSE dilution of proliferating cells was made. The FACS results are depicted in FIGS. 3C and 3D, showing that the CAR-A*02 was strongly activated by HLA-A*02 positive PBMC, resulting in a strong proliferation and up-regulation of the CD39 effector molecule. This effect was much stronger than in the activated Treg cells expressing the control CAR. The Treg cells expressing the control CAR are likely activated via their allospecific TCR, as this is found on up to 12% of all nTreg cells. The up-regulation of CD39 was also found upon activation using the combination of anti-CD3 and anti-CD28 antibodies, which act on the TCR. These data indicate that the Treg cells expressing the CAR-A*02 according to the invention can be activated equally well via the CAR-A*02 or via the TCR.

It is assumed that the high proliferative capacity of the Treg cells expressing the CAR-A*02 according to the invention after transfer into a patient supports their effect, e.g. their niche filling capability.

### Example 2: Suppressor Activity of CAR-A*02 In Vitro

The suppressor activity of Treg cells expressing the CAR-A*02 of Example 1 was tested by assaying the suppression of an allogeneic mixed lymphocyte reaction (MLR) directed against HLA-A*02 positive CD1c stimulator cells. The responder cells were CFSE labelled (5 mM) isolated CD4+CD25− effector T-cells that were co-cultured with isolated HLA-A*02 CD1c+ cells in the presence of various ratios of syngeneic HLA-A1 CD4⁺CD25⁺CD127^{low} Treg (nTreg) cells or syngeneic Treg cells expressing the CAR-A*02 for five days. Suppression of syngeneic effector T-cell proliferation was calculated on the basis of the ratios Treg/Teff via a CFSE dilution assay. For comparison, non-transduced nTreg cells from the same transduction experiment were used.

The result is depicted in FIG. 3E, showing that the Treg cells expressing the CAR-A*02 (A2-CAR Tregs) much more potently inhibited the proliferation of allospecific effector T-cells compared to the non-transduced Treg cells (nTregs). The Treg cells expressing the CAR-A*02 were more potent suppressors at almost all ratios of the CAR-A*02-expressing Treg/effector T-cells tested. Even at a ratio of 1:64 of CAR-A*02-expressing Treg/effector T-cells (Ratio Treg/Teff), inhibition of over 60% was observed, demonstrating the strong suppressive activity conferred by the CAR-A*02 fusion protein.

For analysis of the consequences of signalling in Treg cells by CAR-A*02, a transcriptome analysis comprising 1149 genes was made by deep sequencing in non-activated and CAR-A*02 expressing Treg cells and compared to the results obtained for non-transduced Treg cells. The CAR-A*02 expressing Treg cells (CD4⁺CD25^{high}CD127^{low}R) were activated with irradiated (30 Gy) HLA-A*02+ PBMC as stimulator cells by co-culture for 36 h. As a control, non-transduced Treg cells were left untreated or were stimulated via their TCR by the combined anti-CD3/anti-CD28 antibodies, for 48 h. After stimulation, RNA was isolated using the MicroRNeasy kit (obtained from Qiagen), quality and integrity of total RNA was measured on an Agilent Technologies 2100 Bioanalyser. An RNA sequencing library was generated from 100 ng total RNA using TruSeq RNA Sample Prep kits v2 (obtained from Illumina) for mRNA purification followed by ScriptSeq v2 RNA Seq Library Preparation kit (obtained from Epicentre) according to the manufacturer's protocols. The libraries were sequenced on an Illumina HiSeq2500 device using TruSeq SBS kit v3-HS (50 cycles, single ended run) with an average of 3×10⁷ readings per RNA sample. Readings were aligned to the reference genome hg19 using the open source short read aligner STAR with default settings. The readings per gene after alignment were made by the feature.count function of the R package termed Rsubread. For log 2 transformation of raw count data followed by data normalisation and statistical determination of differentially expressed genes, the R package termed edgeR was used.

It was found that the CAR-A*02 transduced Treg cells and the non-transduced Treg cells had a very similar pattern of activated and down-regulated transcripts, supporting the notion that signalling via the CAR-A*02 leads to comparable transcriptional profiles in Treg cells as signalling via the TCR. The activation of the CAR-A*02 or of the TCR, respectively, resulted in drastic changes of the transcriptional profiles compared to the non-activated state, but the transcriptional profiles of both activated states were similar to one another. An analysis of specific genes that are involved in Treg cell function and their homing revealed subtle differences. The CAR-A*02 activated Treg cells expressed higher amounts of IL-4, IL-5 and IL-10 but slightly lower transcript numbers of CTLA4 and IL-2R. These decreased transcript levels had no apparent consequence for CTLA4 protein expression (FIG. 2A), nor for IL-2 signalling (FIG. 2B, FIG. 2C).

### Example 3: Suppressor Activity of CAR-A*02 In Vivo

As an example for suppressor activity in vivo, humanized non-obese diabetic (NOD)-RAG1^{null}IL2γ^{null} (NRG) mice, non-reconstituted, received 5×10⁴ CD4⁺CD25⁺CD127^{low} human Treg cells from HLA-A*02 negative donors, which Treg cells were transduced with the retroviral vector encoding the CAR-A*02 according to Example 1, or the same Treg cells transduced with the control CAR (specific for PE) of Example 1, or the non-transduced Treg cells. As an example for transplanted tissue, mice were injected into each ear pinnae with admixed 5×10⁵ irradiated syngeneic HLA-A*01 PBMC and allogeneic irradiated HLA-A*02 PBMC as an in vivo MLR.

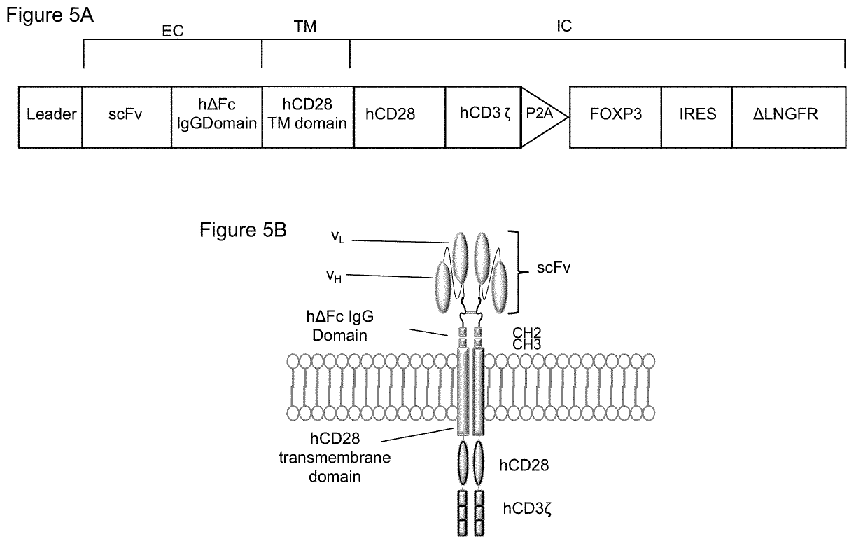

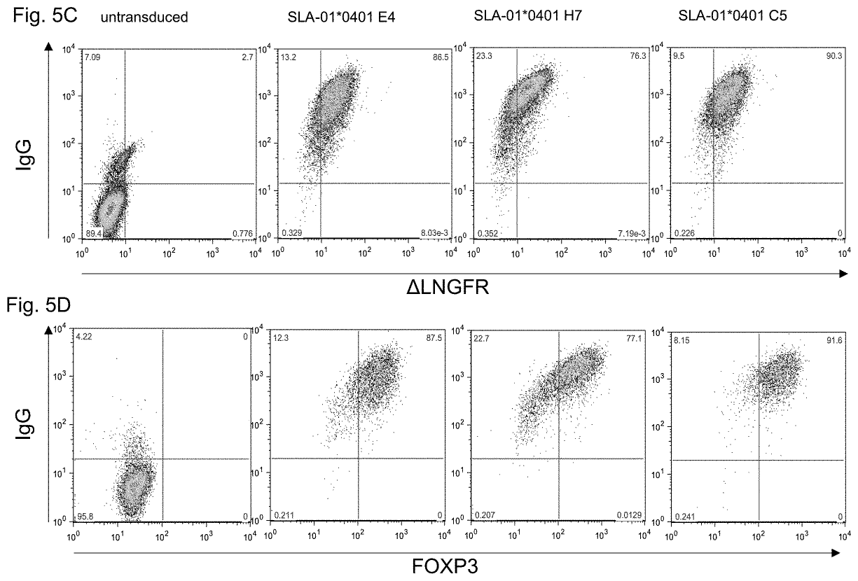

These experiments were performed in a blinded manner. For determining the suppressor activity, ear swelling was measured using a spring-loaded digital thickness gauge. FIG. 5A shows the results of the ear swelling, calculated as the difference between ear thickness prior to injection and 24 h after injection, with each value related to the ear swelling observed in the other ear of the animal that had not been injected with Treg cells as an internal control.

The result is depicted in FIG. 5A, showing a significantly stronger inhibition of the allogeneic mixed lymphocyte reaction for the Treg cells expressing CAR-A*02 (A2-CAR Tregs) in comparison to Treg cells expressing the control CAR (control CAR Tregs) and in comparison to the non-transduced Tregs (CD4+ CD25high Tregs).

In another experiment, the suppressor activity was analysed in immune reconstituted NRG mice. Currently, testing transplant rejection in such mice is difficult, because in immunocompetent mice, an allogeneic skin transplant is rejected within 10 d, while a similar rejection in humanized NRG mice does not occur before day 30 after transplantation. At this late point in time, xenospecific graft-versus-host responses already become evident after immune reconstitution. In order to avoid other effects than the GvHD reaction, a stringent rejection model was used in which allogeneic transplanted cells are completely rejected by day 5 after transplantation by injection. The use of injected allogeneic transplant cells has the additional advantage that homing of Treg cells to transplanted tissues should not play a major role because the immune response is initiated in the spleen, therefore avoiding possible effects of the perturbed homing in humanized mice.

As Treg cells, CD4⁺CD25⁺CD127^{low} human Treg cells transduced with CAR-A*02 according to Example 1, or the same Treg cells transduced with the control CAR (specific for PE) of Example 1, or the non-transduced Treg cells were used. Immune reconstitution was monitored by FACS 14 d after reconstitution by expression of human CD8 and human CD4 in peripheral blood samples from the mandibular vein. Animals with no perceptible reconstitution of human CD8 and CD4 T cells were excluded from experiments. On day 14 after immune reconstitution, mice were injected i.v. with 5×10⁵ syngeneic PBMC labelled with CFSE and 5×10⁵ HLA-A*02 PBMC as allogeneic positive target cells which were labelled with APC proliferation dye. Simultaneously, different animals received different Treg cells at 5×10⁴, which were Treg expressing CAR-A*02 (plus A2-CAR Tregs) or control CAR (plus control CAR Tregs) or non-transduced (plus nTregs). Five days after injection, mice were sacrificed and blood and splenic cells were analysed for allogeneic targets and syngeneic donor cells, and compared to those obtained in animals that did not receive Treg cells (no add. Tregs). The labelling of syngeneic and allogeneic cells allowed to assess the relative killing of allogeneic target cells in the animals as both cell populations were injected at a 1:1 cell ratio.

Representative FACS results are depicted in FIG. 5C, showing that in immunocompetent mice allogeneic target cells were no longer detected after 120 h after transplantation, corresponding to the fast rejection of allogeneic tissue in non-humanized mice. The injection of Tregs expressing the control CAR or of non-transduced Tregs had a small effect in preventing killing of allogeneic target cells, the transfer of Treg cells expressing a CAR-A*02 completely prevented the rejection of allogeneic target cells.

### Example 4: Expression of CAR-SLA-01*0401

Fusion proteins CAR-SLA-01*0401, containing scFv having specificity for the porcine SLA-01*0401, were expressed from a nucleic acid construct according to FIG. 5A, encoding from 5′ to 3′ adjacent to one another a leader peptide for secretion (SEQ ID NO: 25), an scFv specific for the MHC class I, a hΔFc IgG domain as a preferred hinge, the hCD28 TM domain as a transmembrane domain, hCD28, hCD3ζ, P2A, FOXP3, an IRES, and ΔLNGFR as a reporter. Therein, the P2A is arranged such that following expression, FOXP3 is cleaved off and can translocate into the nucleus. The scFv and the hinge form the extracellular (EC) portion, The TM domain forms the transmembrane portion, and the hCD28 and hCD3ζ as well as the optional P2A and FOXP3 form the intracellular (IC) portion. Also, the reporter ΔLNGFR is intracellular. The reporter is expressed from the same nucleic acid construct, indicating its presence, but as a separate protein.

FIG. 5B in a model shows the arrangement of the CAR-SLA-01*0401 with its transmembrane domain spanning a cell membrane and the scFv arranged on the outer cell surface and the intracellular signalling domains arranged within the cytoplasm.

Sc1 cells were transduced separately with nucleic acid constructs encoding fusion proteins according to FIG. 5A of a leader, an scFv, the hΔFc IgG domain of SEQ ID NO: 20, the hCD28 TM domain fused to the hCD28 signalling domain and fused to hCD3ζ of SEQ ID NO: 21, with the optional P2A-hFOXP3 of SEQ ID NO: 22. The reporter ΔLNGFR of SEQ ID NO: 23 was transcribed from an IRES (internal ribosomal entry site). The scFv was one selected from SEQ ID NO: 16 to SEQ ID NO: 19, having specificity for the porcine SLA-01*0401.

FACS results for staining of non-transduced cells for comparison (untransduced) and for transduced cells expressing the CAR-SLA-01*0401 containing the scFv E4 of SEQ ID NO: 17 (SLA-01*0401 E4), the scFv H7 of SEQ ID NO: 19 (SLA-01*0401 H7) or the scFv C5 of SEQ ID NO: 16 (SLA-01*0401 C5) are shown in FIG. 5C with staining for the reporter ΔLNGFR and staining with an anti-IgG-Fab, and in FIG. 5D with staining for FOXP3 and staining with an anti-IgG-Fab. The results show surface expression of the hinge formed by the hΔFc IgG domain and concurrent expression of the reporter and concurrent expression of FOXP3, demonstrating that the fusion protein is located in the cell membrane with the extracellular domains on the outside and the signalling domains on the inside.

The cross-reactivity of the CAR-SLA-01*0401 to human cells was tested using soluble scFv antibodies of E4 (SEQ ID NO: 17), H7 (SEQ ID NO: 19), C5 (SEQ ID NO: 16), and F11 (SEQ ID NO: 18). The soluble scFv contained an additional C-terminal His₆-Tag not given in the sequences.

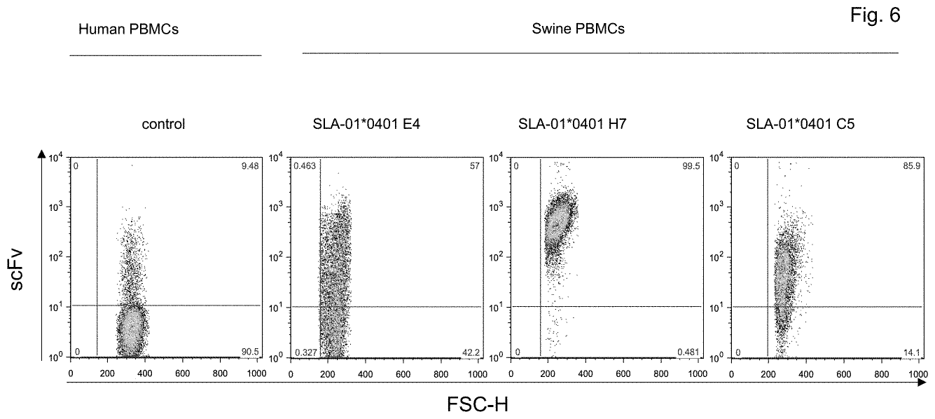

FIG. 6 shows the FACS results for human PBMC and porcine PBMC, respectively, after incubation with the labelled scFv. The results show no cross-reactivity of the scFv with human PBMC and labelling of the porcine PBMC with each of the scFv.

For analysis of the function of CAR-SLA-01*0401-transduced cells, T-cell hybridomas stably expressing a reporter construct containing an NFAT-sensitive IL-2 promoter to control GFP expression were transduced with the CAR-SLA-01*0401. For detection of CAR expression in FACS analysis, the reporter ΔLNGFR was detected, NFAT stimulation was detected as expression of GFP (green fluorescent protein).

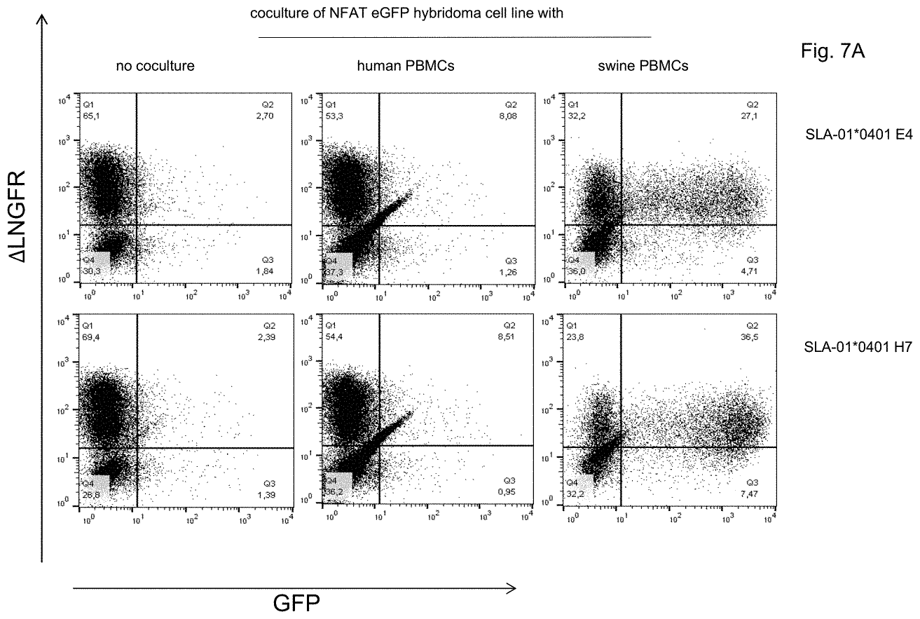

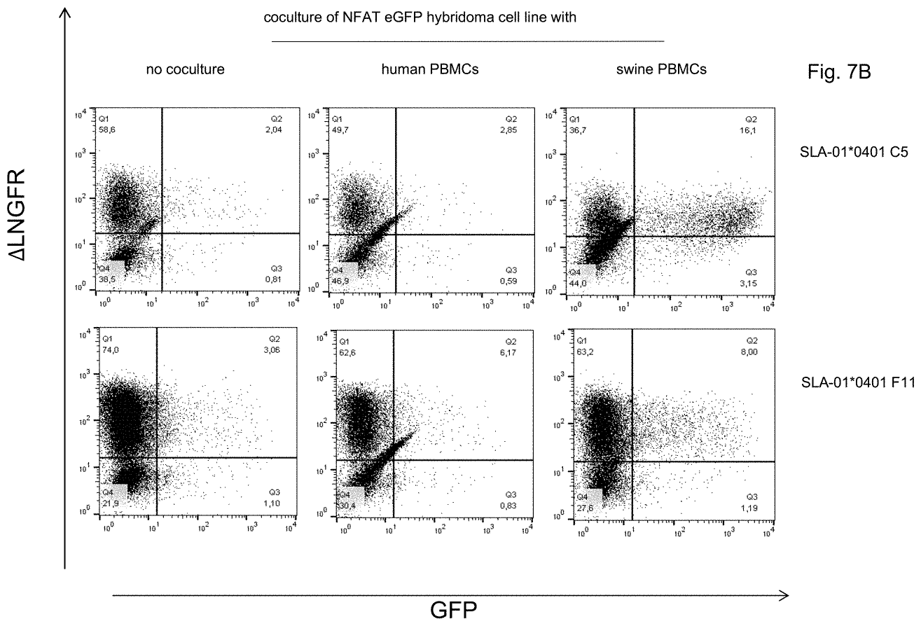

As shown in FIGS. 7A and 7B, it was found that CAR-SLA-01*0401-transduced cells, containing the scFv domain E4, H7, F11 or C5, did not show any NFAT activation nor the associated expression of GFP after transduction, but NFAT activation and GFP expression were strongly up-regulated by co-culture with porcine PBMC acting as stimulator cells but not in response to human PBMC. This result shows that the CAR-SLA-01*0401 according to the invention is capable of signal transduction necessary to activate NFAT in the presence of porcine cells. As the hybridomas do not express any endogenous T-cell receptor (TCR), the signal transduction that was observed is entirely caused by the signalling of the CAR-SLA-01*0401.

### Example 5: Suppressor Activity of CAR-SLA-01*0401 In Vivo

As an example for a solid allogeneic transplant tissue, porcine islet cells were transplanted into mice as recipients. The most commonly used approach to study allogeneic or xenogeneic islet function in preclinical models is to monitor blood glucose levels after islet transplantation into hyperglycemic humanized mice. Presently, animals were treated with streptozozocin (STZ) to render the mice hyperglycemic by the destruction of insulin producing islet cells. Those hyperglycemic mice were transplanted with isolated adult pig islets under the kidney capsule in order to replace destructed murine islet cells. 14 days following the transplantation, animals were reconstituted with human PBMCs (xenogeneic to transplanted pig islets) with additional and without additional (control group) porcine Treg cells transduced to express a CAR-SLA-01*0401. Islet function and cell viability were monitored via blood glucose level of experimental animals. Those data are compared with immunofluorescence data from explanted kidneys at final stage of experiments to explore accumulation of CAR Tregs in islet cell clusters.

1. A human Treg cell expressing a fusion protein which is a chimeric antigen receptor (CAR) which contains an scFv domain specific for human HLA-A*02 (CAR-A*02) and which Treg cell is genetically manipulated to express FOXP3 for use in the treatment of HvG disease.

2. The human Treg cell for use according to claim 1, wherein said cell is genetically manipulated to constitutively express FOXP3.

3. The human Treg cell for use according to claim 1, wherein an expression cassette encoding human FOXP3 is introduced concurrently with a nucleic acid sequence encoding the CAR-A*02 into the Treg cell.

4. The human Treg cell for use according to claim 1, wherein expression of FOXP3 with the fusion protein CAR-A*02 is by expression of a fusion of P2A with C-terminally fused FOXP3 in one unified fusion protein.

5. The human Treg cell for use according to claim 4, wherein the fusion of P2A with FOXP3 has a sequence as set out in SEQ ID NO. 22.

6. The human Treg cell for use according to claim 1, wherein said cell is further genetically manipulated to express a caspase-9 dimeriser system.

7. The human Treg cell for use according to claim 1, wherein the CAR-A*02 comprises or consists of a single-chain variable fragment antibody domain (scFv), a hinge, a transmembrane domain, an intracellular hCD28 signalling domain and an intracellular CD3 signalling domain.

8. The human Treg cell for use according to claim 7, wherein the hinge is a modified hCD8 hinge and wherein the transmembrane domain is a hCD8 transmembrane domain.

9. An in vitro method for introducing suppressor activity specific for HLA-A*02 in Treg cells, by introducing a nucleic acid sequence encoding the fusion protein CAR-A*02 as defined in claim 1 into Treg cells, wherein said cells do not contain or express HLA-A*02, and expressing the fusion protein and further genetically manipulating said cells to express FOXP3.

10. The method of claim 9, wherein the cells are genetically manipulated to constitutively express FOXP3.

11. The method of claim 9, wherein an expression cassette encoding human FOXP3 is introduced concurrently with said nucleic acid sequence encoding the CAR-A*02 into the Treg cell.

12. The method of claim 9, wherein expression of FOXP3 with the fusion protein CAR-A*02 is by expression of a fusion of P2A with C-terminally fused FOXP3 in one unified fusion protein.

13. The method of claim 12, wherein the fusion of P2A with FOXP3 has a sequence as set out in SEQ ID NO. 22.

14. A nucleic acid sequence encoding a fusion protein which is a chimeric antigen receptor (CAR) which contains an scFv domain specific for human HLA-A*02 (CAR-A*02), P2A and a C-terminally fused FOXP3, in one unified fusion protein.

15. A viral vector comprising the nucleic acid of claim 14.


SEQUENCE LISTING (30 sequences)

SEQ ID NO: 1 - protein, 251 aa, Artificial Sequence
  CHAIN 1..123: variable heavy chain
  scFv (KAZ15-C1)
  CHAIN 124..141: linker
  CHAIN 142..251: variable light chain
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGYDSRGSYYYMDVWGKGTTVTVSSGSASAPKLEEGEFSEARVQSVLTQPPSTSGTPGQRVTISCSGSSSNIGGNAVNWYQHFPGTAPTLLIYSNNQRPSGVPERFSGSKSGTSASLTVSGLQAEDEADYYCTAWDDSLRGYLFGTGTKVTVL

SEQ ID NO: 2 - protein, 249 aa, Artificial Sequence
  CHAIN 1..123: variable heavy chain
  scFv (KAZ15-F3)
  CHAIN 124..141: linker
  CHAIN 142..249: variable light chain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDREELLALFGGMDVWGQGTTVTVSSGSASAPKLEEGEFSEARVQPVLTQPSSVSVAPGQTARITCGGNNIGSKSVHWYQQKPGQAPVLVVYDDSDRPSGIPERFSGSNSGNTATLTISRVEARDEADYYCHVWDAKTNHQVFGGGTRLTVQ

SEQ ID NO: 3 - protein, 251 aa, Artificial Sequence
  CHAIN 1..123: variable heavy chain
  scFv (SH1319-A5)
  CHAIN 124..141: linker
  CHAIN 142..251: variable light chain
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARPQSRWLQSGDAFDIWGQGTMVTVSSGSASAPKLEEGEFSEARVQPVLTQPRSVSGSPGQSVTISCTGTSSDVGGYNRVSWYQQTPGTAPKLMIYEVSNRPSGVSNRFSGSKSGNTASLTISGLQAEDEADYYCSSYTSSSTVVFGGGTKLTVL

SEQ ID NO: 4 - protein, 246 aa, Artificial Sequence
  CHAIN 1..119: variable heavy chain
  scFv (SH1313-B8)
  CHAIN 120..137: linker
  CHAIN 138..246: variable light chain
QVQLQQSGAEVKKPGASVKVSCKASGYTFTSYAMHWVRQAPGQRLEWMGRINAGNGNTKYSQKFQGRVTITRDTSASTAYMELSSLRSEDTAVYYCARDLTGTLLFDYWGQGTLVTVSSGSASAPKLEEGEFSEARVQSVLTQPPSASGTPGQRVTISCSGSSSNIGSNGVKWYQQLPGTAPKLVIYRDYQRPSGVPDRFSGSKSGTSASLAISGLQSEDEAKYYCAAWDDSLNVVFGGGTQLTVL

SEQ ID NO: 5 - protein, 251 aa, Artificial Sequence
  CHAIN 1..123: variable heavy chain
  scFv (SH1319-B11)
  CHAIN 124..141: linker
  CHAIN 142..251: variable light chain
QVQLVQSGAEVKKPGSSVKVSCKASGGTFSSYAISWVRQAPGQGLEWMGGIIPIFGTANYAQKFQGRATITADESTSTAYMELSSLRSEDTAVYYCARRAERWLHLSGAFDIWGQGTMVTVSSGSASAPKLEEGEFSEARVQPVLTQSSSASGTPGQRVAISCSGSSSNVGSNTVNWYQQSPGTAPKLLISSNHQRPSGVPDRFSGSKFGTSASLAISGLQSEDEADYYCGAWDDSLNGYVFGSGTKVTVL

SEQ ID NO: 6 - protein, 244 aa, Artificial Sequence
  CHAIN 1..116: variable heavy chain
  scFv (SH1319-C5)
  CHAIN 117..134: linker
  CHAIN 135..244: variable light chain
QVQLVQSGGGLVQPGGSLRLSCAASGFTFSSYAMSWVRQAPGKGLEWVSAISGSGGSTYYADSVKGRFTMSRDNAKNSLYLQMNSLRVEDSAVYYCATGHYGDYVWGQGALVTVSSGSASAPKLEEGEFSEARVQAGLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGGGTKLTVL

SEQ ID NO: 7 - protein, 246 aa, Artificial Sequence
  CHAIN 1..118: variable heavy chain
  scFv (SH1319-D4)
  CHAIN 119..136: linker
  CHAIN 137..246: variable light chain
QVQLQQSGAEVKKPGASVKVSCKASGYTFTSYAMHWVRQAPGQRLEWMGWINAGNGNTKYSQKFQGRVTITRDTSASTAYMELSSLRSEDTAVYYCARVSSGGAFDIWGQGTVVTVSSGSASAPKLEEGEFSEARVQSALTQPASVSGSPGQSITISCTGTGSDVGGYKYVSWYQHHPGKAPRLIIYDVNYWPSGVSHRFSGSKSGNTASLTISGLQSEDEADYYCSSYRTGDTWVFGGGTKLTVL

SEQ ID NO: 8 - protein, 248 aa, Artificial Sequence
  CHAIN 1..123: variable heavy chain
  scFv (SH1319-D8)
  CHAIN 124..141: linker
  CHAIN 142..248: variable light chain
QVQLVQSGAEVKKPGSSVKVSCKASGGTFSSYGFSWVRQAPGQGLEWMGEIIPMFGTANYAQKLQGRVTITAETSTSTVYMELSSLRSEDTATYYCARVPRSSSGYNYGMDVWGQGTTVTVSSGSASAPKLEEGEFSEARVDIQMTQSPSSLSASVGDRVTITCRASQGISNYLAWYQQKPGKVPKLLIYAASTLQSGVPSRFSGSGSGTDFTLTISSLQPEDVATYYCQKYNSAPRTFGQGTKVEIK

SEQ ID NO: 9 - protein, 250 aa, Artificial Sequence
  CHAIN 1..122: variable heavy chain
  scFv (SH1319-E4)
  CHAIN 123..140: linker
  CHAIN 141..250: variable light chain
QVQLQQSGPGLLKPSQTLSLTCAVSGDSVSTNSGAWSWIRQSPSRGLEWLGRTYYRSKWSTDYALSLQSRVTIKSDRSKNQFSLQLDSVTPEDTAIYYCARENWNSGGFDYWGQGTLVTVPSGSASAPKLEEGEFSEARVQPVLTQSSSASGSPGQSVTISCTGTSSDVGGYNYVSWYQQHPGKAPKLMIYEVSKRPSGVPDRFSGSKSGSTASLTVSGLQAEDEAEYYCSSYAGSNNYVFGTGTKVTVL

SEQ ID NO: 10 - protein, 249 aa, Artificial Sequence
  CHAIN 1..122: variable heavy chain
  scFv (SH1319-E12)
  CHAIN 123..140: linker
  CHAIN 141..249: variable light chain
EVQLVESGAEVKKPGSSVKVSCKASGGTFSSYAISWVRQAPGQGLEWMGGIIPIFGTANYAQKFQGRVTITADKSTSTAYMELSSLRSEDTAVYYCARAASRWEPGDAFDIWGQGTMVTVSSGSASAPKLEEGEFSEARVQPVLTQSSSVSVAPGKTARVTCGGDNIGGKSVHWYQQRAGQAPVLVISHDTDRPSGIPERFSGSKSGTSASLAISGLRSEDEADYYCAVWDASLGGSWLFGGGTKLTVL

SEQ ID NO: 11 - protein, 252 aa, Artificial Sequence
  CHAIN 1..122: variable heavy chain
  scFv (SH1319-F6)
  CHAIN 123..141: linker
  CHAIN 142..252: variable light chain
QVQLVQSGAEVKKPGASVKVSCKTSGYTFTSYDISWVRQAPGQGLEWMGWISAYNGNTNYAQKLQGRVTMTTDTSTSTAYMELRRLRSDDTAVYYCARGGRWLRSASSFDYWGQGTLVTVSSGSASAPKLEEGEFSEARVQAGLTQPPSVSGAPGQRVTISCTGSSSNIGAAYDVHWYQQLPGAAPKLLIFGDSNRPSGVPDRFSGSKSDTSASLAITGLQAEDEADYYCQSFDSSLSGSRVFGGGTKLTVL

SEQ ID NO: 12 - protein, 251 aa, Artificial Sequence
  CHAIN 1..123: variable heavy chain
  scFv (SH1319-G3)
  CHAIN 124..141: linker
  CHAIN 142..251: variable light chain
EVQLVESGGGLVKPGGSLRLSCAASGFTFSDHYMSWVRQAPGKGLEWVSYITSGGSSIYYADSVKGRFTISRDNAKNSLYLQMNSLRAEDTAVYYCARGLDSSAYQGRAFDIWGQGTMVTVSSGSASAPKLEEGEFSEARVLPVLTQPPSASGTPGQRVTISCSGSSSNIGSNPVHWYQQLPGTAPKLLVYRNNQRPSGVPDRFSGSKSGTSASLAISGLRSEDEADYYCAAWDVSLSGVVFGGGTKLTVL

SEQ ID NO: 13 - protein, 81 aa, Artificial Sequence
  CD8 hinge domain and transmembran domain
FVPVFLPAKPTTTPAPRPPTPAPTIASQPLSLRPEASRPAAGGAVHTRGLDFADIYIWAPLAGTCGVLLLSLVITLYCNHR

SEQ ID NO: 14 - protein, 42 aa, Artificial Sequence
  CD28 signalling domain
WVRSKRSRLLHSDYMNMTPRRPGPTRKHYQPYAPPRDFAAYR

SEQ ID NO: 15 - protein, 113 aa, Artificial Sequence
  CD3zeta signalling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRS

SEQ ID NO: 16 - protein, 306 aa, Artificial Sequence
  scFv (C5)
IKEEKLTMKYLLPTAAAGLLLLAAQPAMAQVQLQESGGGVIQPGRSLRLSCAASGFTFSGHGFHWVRQAPGKGLEWVAVIWYDGSKTFYADSVKGRFTISRDNSRNTVFLQMNSLRAEDTAVYYCARDLSYYAMDVRGQGTTVTVSSGSASAPKLEEGEFSEARVQTVVTQEPSLTVSPGGTVTLTCASSTGAVTSGYYPNWFQQKPGQAPRALIYSTSNKHSWTPARFSGSLLGGKAALTLSGVRPEDEAEYYCLLYYGGARVFGGGTKLTVLGQPKAAPSVTLFPPSSAAAGSEQKLISEEDLS

SEQ ID NO: 17 - protein, 315 aa, Artificial Sequence
  scFv (E4)
IKEEKLTMKYLLPTAAAGLLLLAAQPAMAQMQLVQSGAEVKKPGSSVKVSCKASGGTFSSYAISWVRQAPGQGLEWMGGIIPIFGTADYAQKFQGRVTITADESTSTAYMELSSLRSEDTAVYYCARVGPTSSSSYYYGMDVWGQGTTVTVSSGSASAPKLEEGEFSEARVQAVLTQPPSVSGAPGQRVTISCTGSSSNIGAGYDVHWYQQLPGTAPKLLIYGNSNRPSGVPDRFSGSKFGTSASLAITGLQAEDEADYYCQSFDTSLSGSKVFGGGTKLTVLGQPKAAPSVTLFPPSSAAAGSEQKLISEEDLS

SEQ ID NO: 18 - protein, 307 aa, Artificial Sequence
  scFv (F11)
MKYLLPTAAAGLLLLAAQPAMAQVQLVQSGGGVVQPGRSLRLSCAASGFTFSSYGMHWVRQAPGKGLEWVAVISYDGRNVHYADSVKGRFTISRDNSKNTLHLQMNSLRPEDSALYYCVKDLARVVITPGGMHVWGQGTTVTVSSGSAFAPKLEEGEFSEARVQAVVTQEPSLSVSPGGTVTLTCASSTGTVTGASSPNWFQQKPGQVPRPLIYGTTNKHSWTPARFSGSLLGGKAALTVSGVQPEDEAEYYCLLYSGGGQPYWMFGGGTKLTVLGQPKAAPSVTLFPPSSAAAGSEQKLISEEDLS

SEQ ID NO: 19 - protein, 310 aa, Artificial Sequence
  scFv (H7)
KEEKLTMKYLLPTAAAGLLLLAAQPAMAQVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKEAYNWNDGGWFDPWGQGTLVTVSSGSASAPKLEEGEFSEARVQAVLTQPPSTSGTPGQGVTISCSGSRSNIGPNYVHWYQQLPGAAPKVLIYRNYQRPSGVPDRISASKSGTSASLAISGLRSEDEAVYYCASWDDTLGAVVFGGGTKLTVLGQPKAAPSVTLFPPSSAAAGSEQKLISEEDLS

SEQ ID NO: 20 - protein, 280 aa, Artificial Sequence
  hFC IgG domain
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIL

SEQ ID NO: 21 - protein, 184 aa, Artificial Sequence
  hCD28 transmembrane domain - hCD3zeta signalling domain
DPKFWVLVVVGGVLACYSLLVTVAFIIFWVRSKRSRLLHSDYMNMTPRRPGPTRKHYQAYAAARDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 452 aa, Artificial Sequence
  fusion P2A hFOXP3
GATNFSLLKQAGDVEENPGPSMPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 23 - protein, 280 aa, Artificial Sequence; identical to SEQ ID NO: 20
  LNGFR (reporter)

SEQ ID NO: 24 - protein, 21 aa, Artificial Sequence
  secretory leader peptide
MALPVTALLLPLALLLHAAAP

SEQ ID NO: 25 - protein, 25 aa, Artificial Sequence
  secretory leader peptide
MDFQVQIFSFLLISASVIMSRGVHS

SEQ ID NO: 26 - protein, 246 aa, Artificial Sequence
  CHAIN 1..123: heavy chain variable region
  scFv (ELFA*2)
  CHAIN 26..33: CDR1
  CHAIN 51..58: CDR2
  CHAIN 97..112: CDR3
  CHAIN 124..139: linker
  CHAIN 140..246: light chain variable region
  CHAIN 166..171: CDR1
  CHAIN 189..191: CDR2
  CHAIN 228..237: CDR3
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 27 - protein, 21 aa, Artificial Sequence
  CD28 extracellular domain signal peptide
MALPVTALLLPLALLLHAAAP

SEQ ID NO: 28 - protein, 245 aa, Artificial Sequence
  CHAIN 1..123: heavy chain variable region
  variant ELFA*2
  CHAIN 124..138: linker
  CHAIN 139..245: light chain variable kappa
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 29 - DNA, 368 bp, Artificial Sequence
  coding sequence for heavy chain of scFv of CAR
caggtgcagctggtgcagtctgggggaggcgtggtccagcctggggggtccctgagagtctcctgtgcagcgtctggagtcaccctcagtgattatggcatgcattgggtccgccaggctccaggcaaggggctggagtgggtggcttttatacggaatgatggaagtgataaatattatgcagactccgtgaagggccgattcaccatctccagagacaactccgagaaaacagtgtctctgcaaatgagcagtctcagagctgaagacacggctgtgtattactgtgcgaaaaatggcgaatctgggcctttggactactggtacctcgatctctggggccgtggcaccctggtcaccgtgtcgag

SEQ ID NO: 30 - DNA, 324 bp, Artificial Sequence
  coding sequence for light chain of scFv of CAR
gatgttgtgatgactcagtctccatcctccctgtctgcatctgtaggagacagagtcaccatcacttgccagtcgagtctggacattagccactatttaaattggtatcagcagaaaccagggaaagcccctaagctcctgatctacgatgcatccaatttggaaacaggggtcccgtcaaggttcagtggaagtggatctgggacacactttactttcaccatcagcagcctgcagcctgaagattttgcaacatattactgtcaacagtatgataatctccccctcactttcggcggagggaccaagctggagatcaaacgt